(19) United States
(12) Patent Application Publication
(10) Pub. No.: US 2021/0179666 A1
(43) Pub. Date: Jun. 17, 2021

(54) CYCLIC PEPTIDES AND METHODS OF USE THEREOF

(71) Applicant: Regents of the University of Minnesota, Minneapolis, MN (US)

(72) Inventors: Carrie Haskell-Luevano, Minneapolis, MN (US); Mark David Ericson, Minneapolis, MN (US); Zoe Margaret Koerperich, Minneapolis, MN (US)

(73) Assignee: Regents of the University of Minnesota, Minneapolis, MN (US)

(21) Appl. No.: 17/113,887

(22) Filed: Dec. 7, 2020

Related U.S. Application Data
(60) Provisional application No. 62/945,698, filed on Dec. 9, 2019.

(51) Int. Cl. C07K 7/64

(52) U.S. Cl. CPC C07K 7/64; A61K 38/00

(57) ABSTRACT

Certain embodiments of the invention provide a cyclic compound of formula I:

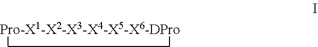

wherein Pro, DPro, X¹, X², X³, X⁴, X⁵, and X⁶ are defined as described herein; or a salt thereof. Certain embodiments also provide compositions comprising such compounds, as well as methods of using such compounds and compositions.

### CROSS-REFERENCE TO RELATED APPLICATION

This application claims priority to U.S. Provisional Application No. 62/945,698 that was filed on Dec. 9, 2019. The entire content of the application referenced above is hereby incorporated by reference herein.

### GOVERNMENT FUNDING

This invention was made with government support under DK108893 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 15, 2021, is named 09531_507US1_SL.txt and is 16,219 bytes in size.

### BACKGROUND OF THE INVENTION

The melanocortin system has been associated with many physiological functions, including skin pigmentation, steroidogenesis, and energy homeostasis. Five melanocortin receptors have been identified to date that are members of the super-family of G protein-coupled receptors (GPCRs). The melanocortin receptors couple to G_{αs} protein subunits and increase intracellular levels of cAMP following agonist stimulation. Naturally occurring ligands for the receptor include peptide agonists derived from the proopiomelanocortin gene transcript and two endogenous antagonists, agouti signaling protein (ASIP) and agouti-related protein (AGRP). While the melanocortin-5 receptor (MC5R) has been implicated in metabolic disorders, acne, inflammation, mental disorders and stress, the physiological functions of MC5R remain to be elucidated. Investigating biological pathways and disorders associated with MC5R may result in new therapeutic agents.

Accordingly, there is a need for new ligands (e.g., selective ligands) for the melanocortin receptors, including MC5R.

### SUMMARY OF THE INVENTION

Certain embodiments of the invention provide a cyclic compound of formula I:

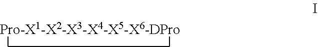

wherein:

Pro is a residue of L-proline;

X¹ is a residue of Arg, NArg, His or Cys;

X² is a residue of Phe, NPhe or Tyr;

X³ is a residue of Phe or NPhe;

X⁴ is a residue of Asn, Dap or NDab;

X⁵ is a residue of Ala, NAla or Val:

X⁶ is a residue of Phe or NPhe; and

DPro is a residue of D-proline;

or a salt thereof,

provided the compound of formula I is not c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2).

Certain embodiments of the invention provide a pharmaceutical composition comprising a compound of formula I as described, or a pharmaceutically acceptable salt thereof, and a pharmaceutically acceptable carrier.

Certain embodiments of the invention provide a method of modulating the activity of a melanocortin receptor in vitro or in vivo comprising contacting the receptor with an effective amount of a compound of formula I as described herein, or a pharmaceutically acceptable salt thereof.

Certain embodiments of the invention provide a method of modulating metabolic activity and/or modulating appetite in an animal in need thereof, comprising administering to the animal an effective amount of a compound of formula I as described herein, or a pharmaceutically acceptable salt thereof.

Certain embodiments of the invention provide a compound of formula I as described herein, or a pharmaceutically acceptable salt thereof, for modulating metabolic activity and/or modulating appetite in an animal in need thereof.

Certain embodiments of the invention relate to the use of compound of formula I as described herein, or a pharmaceutically acceptable salt thereof, to prepare a medicament for modulating metabolic activity and/or modulating appetite in an animal in need thereof.

Certain embodiments of the invention provide a method for treating cachaxia or a disease associated with cachaxia in an animal in need thereof, comprising administering to the animal a compound of formula I as described herein, or a pharmaceutically acceptable salt thereof.

Certain embodiments of the invention provide a compound of formula I as described herein, or a pharmaceutically acceptable salt thereof, for treating cachaxia or a disease associated with cachaxia in an animal in need thereof.

Certain embodiments of the invention relate to the use of compound of formula I as described herein, or a pharmaceutically acceptable salt thereof, to prepare a medicament for treating cachaxia or a disease associated with cachaxia in an animal in need thereof.

Certain embodiments of the invention provide a method of antagonizing MCR5 in an animal in need thereof, comprising administering to the animal an effective amount of a compound of formula I as described herein, SEQ ID NO:1, SEQ ID NO:2, or a pharmaceutically acceptable salt thereof.

Certain embodiments of the invention provide a compound of formula I as described herein, SEQ ID NO:1, SEQ ID NO:2, or a pharmaceutically acceptable salt thereof, for antagonizing MCR5 in an animal in need thereof.

Certain embodiments of the invention relate to the use of compound of formula I as described herein, SEQ ID NO:1, SEQ ID NO:2, or a pharmaceutically acceptable salt thereof, to prepare a medicament for antagonizing MCR5 in an animal in need thereof.

Certain embodiments of the invention provide a method for treating a disease or disorder associated with excessive MC5R activity in an animal in need thereof, comprising administering to the animal a compound of formula I as described herein, SEQ ID NO:1, SEQ ID NO:2, or a pharmaceutically acceptable salt thereof.

Certain embodiments of the invention provide a compound of formula I as described herein, SEQ ID NO:1, SEQ ID NO:2, or a pharmaceutically acceptable salt thereof, for treating a disease or disorder associated with excessive MC5R activity.

Certain embodiments of the invention relate to the use of a compound of formula I as described herein, SEQ ID NO:1, SEQ ID NO:2, or a pharmaceutically acceptable salt thereof, to prepare a medicament for treating a disease or disorder associated with excessive MC5R activity.

### BRIEF DESCRIPTION OF THE FIGURES

FIG. 1. Illustration of the proposed binding loop of AgRP, with residues Arg-Phe-Phe shown in the upper left quadrant of the FIG. 1 (see, PDB ID: 1HYK for AgRP (87-132)).

FIG. 2. SNPs in the purported binding loop of AgRP, deposited into the NIH Variation Viewer as of November 2017 (www.ncbi.nlm.nih.gov/variation/view/). FIG. 2 discloses SEQ ID NO: 43.

FIG. 3. Illustrations of the in vitro antagonist pharmacology of select orthosteric compounds at the mMC5R. The data has been normalized to basal and NDP-MSH values in the cAMP accumulation assay. A Schild antagonist experimental design was applied and the agonist NDP-MSH was utilized in these experiments. Data points represent values from at least three independent experiments and the error bars represent the standard error of the mean (SEM).

FIG. 4. Illustration of the agonist pharmacology observed for ZMK2-85, ZMK3-18, and ZMK3-20 at the mMC1R, as compared to the control agonist NDP-MSH. Data points represent values from three independent experiments and error bars represent the standard error of the mean (SEM).

FIG. 5. Structures of NArg. NPhe. NDab and DAla.

FIG. 6. Illustrations of the in vitro pharmacology of selected compounds at the mMC1R and mMC5R. The data has been normalized to basal and NDP-MSH values in the cAMP accumulation assay.

FIG. 7. Illustrations of the in vitro pharmacology of MED9-93c at the mMC3R, mMC4R and mMC5R. The data has been normalized to basal and NDP-MSH values in the cAMP accumulation assay.

### DETAILED DESCRIPTION

Certain embodiments of the invention provide a cyclic compound of formula I:

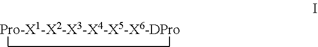

wherein:

Pro is a residue of L-proline, wherein the pyrrolidinyl ring is optionally substituted with one or more halo groups, (C₁-C₄)alkyl, —O(C₁-C₄)alkyl, (C₁-C₄)haloalkyl, or —O(C₁-C₄)haloalkyl;

X¹ is a residue of Arg, NArg, His or Cys;

X² is a residue of Phe, NPhe or Tyr;

X³ is a residue of Phe or NPhe;

X⁴ is a residue of a natural amino acid or an unnatural amino acid;

X⁵ is a residue of Ala, NAla, or Val;

X⁶ is a residue of Phe or NPhe; and

DPro is a residue of D-proline, wherein the pyrrolidinyl ring is optionally substituted with one or more halo groups, (C₁-C₄)alkyl, —O(C₁-C₄)alkyl, (C₁-C₄)haloalkyl, or —O(C₁-C₄)haloalkyl;

or a salt thereof.

Certain embodiments of the invention provide a cyclic compound of formula I:

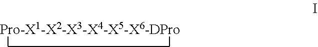

wherein:

Pro is a residue of L-proline;

X¹ is a residue of Arg, NArg, His or Cys;

X² is a residue of Phe, NPhe or Tyr;

X³ is a residue of Phe or NPhe;

X⁴ is a residue of Asn, Dap or NDab;

X⁵ is a residue of Ala. NAla, or Val;

X⁶ is a residue of Phe or NPhe; and

DPro is a residue of D-proline;

or a salt thereof.

Certain embodiments of the invention provide a cyclic compound of formula I:

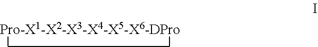

wherein:

Pro is a residue of L-proline;

X¹ is a residue of Arg:

X² is a residue of Phe or Tyr;

X³ is a residue of Phe or NPhe;

X⁴ is a residue of Asn or Dap;

X⁵ is a residue of Ala or Val:

X⁶ is a residue of Phe; and

DPro is a residue of D-proline;

or a salt thereof.

In certain embodiments, the compound of formula I is not c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2).

Thus, certain embodiments of the invention provide a cyclic compound of formula I:

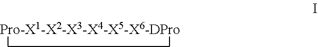

wherein:

Pro is a residue of L-proline;

X¹ is a residue of Arg, NArg, His or Cys;

X² is a residue of Phe, NPhe or Tyr;

X³ is a residue of Phe or NPhe;

X⁴ is a residue of Asn, Dap or NDab;

X⁵ is a residue of Ala, NAla, or Val;

X⁶ is a residue of Phe or NPhe; and

DPro is a residue of D-proline;

or a salt thereof,

provided the compound of formula I is not c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2).

Certain embodiments provide cyclic compound of formula I:

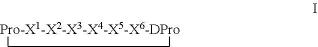

wherein:

Pro is a residue of L-proline;

X¹ is a residue of Arg;

X² is a residue of Phe or Tyr;

X³ is a residue of Phe or NPhe;

X⁴ is a residue of Asn or Dap;

X⁵ is a residue of Ala or Val:

X⁶ is a residue of Phe; and

DPro is a residue of D-proline;

or a salt thereof,

provided the compound of formula I is not c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2).

Certain embodiments provide cyclic compound of formula I:

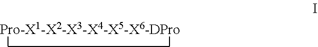

wherein:

Pro is a residue of L-proline;

X¹ is a residue of Arg;

X² is a residue of Phe or Tyr;

X³ is a residue of Phe;

X⁴ is a residue of Asn or Dap;

X⁵ is a residue of Ala or Val;

X⁶ is a residue of Phe; and

DPro is a residue of D-proline;

or a salt thereof,

provided the compound of formula I is not c[Pro-Arg-Phe-Phe-Asn-Ala-Phe-DPro] (SEQ ID NO.1) or c[Pro-Arg-Phe-Phe-Dap-Ala-Phe-DPro] (SEQ ID NO:2).

In one embodiment, Pro is a residue of L-proline. In one embodiment, Pro is a residue of L-proline, wherein the pyrrolidinyl ring is substituted with one or more halo groups, (C₁-C₄)alkyl, —O(C₁-C₄)alkyl, (C₁-C₄)haloalkyl, or —O(C₁-C₄)haloalkyl.

In one embodiment, DPro is a residue of D-proline. In one embodiment, DPro is a residue of D-proline, wherein the pyrrolidinyl ring is substituted with one or more halo groups, (C₁-C₄)alkyl, —O(C₁-C₄)alkyl, (C₁-C₄)haloalkyl, or —O(C₁-C₄)haloalkyl.

In one embodiment, Pro is a residue of L-proline and DPro is a residue of D-proline.

In one embodiment, X¹ is a residue of Arg, NArg, His or Cys. In one embodiment, X¹ is a residue of Arg. In one embodiment, X¹ is a residue of NArg. In one embodiment, X¹ is a residue of His. In one embodiment, X¹ is a residue of Cys.

In one embodiment, X² is a residue of Phe, NPhe or Tyr. In one embodiment, X² is a residue of Phe. In one embodiment, X² is a residue of NPhe. In one embodiment, X² is a residue of Tyr.

In one embodiment, X³ is a residue of Phe or NPhe. In one embodiment, X³ is a residue of Phe. In one embodiment, X³ is a residue of NPhe.

In one embodiment, X⁴ is a residue of a natural amino acid or an unnatural amino acid. In one embodiment. X⁴ is a residue of a natural amino acid. In one embodiment, X⁴ is a residue of an unnatural amino acid. In one embodiment, X⁴ is a residue of an amino acid selected from the group consisting of L-Ala, L-Asp, L-Glu, L-Phe, L-Gly, L-His, L-Ile, L-Lys, L-Leu, L-Met, L-Asn, L-Pro, L-Gln, L-Arg, L-Ser, L-Thr, L-Val, L-Trp, L-Tyr, L-Dap. D-Ala, D-Asp, D-Glu, D-Phe. D-His, D-Ile, D-Lys, D-Leu, D-Met, D-Asn, D-Pro, D-Gln, D-Arg, D-Ser, D-Thr, D-Val, D-Trp, D-Tyr, D-Dap, L-Nle, D-Nle, L-Cha, D-Cha, L-PyrAla, D-PyrAla, L-ThiAla, D-ThiAla, L-Tic, D-Tic, (pCl)L-Phe, (pCl)D-Phe, (pCl)L-Phe, (pI)D-Phe, (pNO2)L-Phe, (pNO2)D-Phe, 2-L-Nal, 2-D-Nal, β-Ala, ε-Aminocaproic acid, Met[O2], dehydPro, and (3I)Tyr. In one embodiment, X⁴ is a residue of Asn, Dap, Ala, Abu, Ser, Thr, Asp, Glu, NDab, His, Nie, Leu, Val, Phe, Trp or Arg. In one embodiment, X⁴ is a residue of DDap, Ser, Abu, Asp or Val.

In one embodiment, X⁴ is a residue of Asn, Dap or NDab. In one embodiment, X⁴ is a residue of Asn. In one embodiment, X⁴ is a residue of Dap. In one embodiment, X⁴ is a residue of NDab.

In one embodiment, X⁵ is a residue of Ala, NAla, or Val. In one embodiment, X⁵ is a residue of Ala. In one embodiment, X⁵ is a residue of NAla. In one embodiment, X⁵ is a residue of Val.

In one embodiment, X⁶ is a residue of Phe or NPhe. In one embodiment, X⁶ is a residue of Phe. In one embodiment, X⁶ is a residue of NPhe.

In one embodiment, X¹ is Arg; X² is Phe or Tyr; X³ is Phe or NPhe; X⁴ is Asn or Dap; X⁵ is Ala or Val; and X⁶ is Phe.

In one embodiment, X¹ is Arg; X² is Phe; X³ is Phe or NPhe; X⁴ is Asn or Dap; X⁵ is Ala or Val; and X⁶ is Phe.

In one embodiment, X¹ is Arg; X² is Tyr; X³ is Phe or NPhe; X⁴ is Asn or Dap; X⁵ is Ala or Val; and X⁶ is Phe.

In one embodiment, X¹ is Arg; X² is Phe or Tyr; X³ is Phe; X⁴ is Asn or Dap; X⁵ is Ala or Val; and X⁶ is Phe.

In one embodiment, X¹ is Arg; X² is Phe or Tyr; X³ is NPhe; X⁴ is Asn or Dap; X⁵ is Ala or Val, and X⁶ is Phe.

In one embodiment, X¹ is Arg; X² is Phe or Tyr; X³ is Phe or NPhe; X⁴ is Asn; X⁵ is Ala or Val; and X⁶ is Phe.

In one embodiment, X¹ is Arg; X² is Phe or Tyr; X³ is Phe or NPhe; X⁴ is Dap; X⁵ is Ala or Val; and X⁶ is Phe.

In one embodiment, X¹ is Arg; X² is Phe or Tyr; X³ is Phe or NPhe; X⁴ is Asn or Dap; X⁵ is Ala; and X⁶ is Phe.

In one embodiment, X¹ is Arg; X² is Phe or Tyr; X³ is Phe or NPhe; X⁴ is Asn or Dap; X⁵ is Val; and X⁶ is Phe.

In one embodiment, the compound of invention is selected from the group consisting of:

(SEQ ID NO: 3)
c[Pro-Arg-Phe-Phe-Asn-Val-Phe-DPro]
(SEQ ID NO: 4)
c[Pro-His-Phe-Phe-Asn-Ala-Phe-DPro]
(SEQ ID NO: 5)
c[Pro-Arg-Tyr-Phe-Asn-Ala-Phe-DPro]
(SEQ ID NO: 6)
c[Pro-Cys-Phe-Phe-Asn-Ala-Phe-DPro]
(SEQ ID NO: 7)
c[Pro-Arg-Phe-Phe-Dap-Val-Phe-DPro]
(SEQ ID NO: 8)
c[Pro-His-Phe-Phe-Dap-Ala-Phe-DPro]
(SEQ ID NO: 9)
c[Pro-Arg-Tyr-Phe-Dap-Ala-Phe-DPro]
(SEQ ID NO: 10)
c[Pro-Cys-Phe-Phe-Dap-Ala-Phe-DPro]
(SEQ ID NO: 11)
c[Pro-Arg-Phe-NPhe-Asn-Ala-Phe-DPro]
(SEQ ID NO: 12)
c[Pro-Arg-Phe-NPhe-Dap-Ala-Phe-DPro]
(SEQ ID NO: 23)
c[Pro-NArg-Phe-Phe-Dap-Ala-Phe-DPro]
(SEQ ID NO: 24)
c[Pro-Arg-NPhe-Phe-Dap-Ala-Phe-DPro]
(SEQ ID NO: 25)
c[Pro-Arg-Phe-Phe-Dap-NAla-The-DPro]
(SEQ ID NO: 26)
c[Pro-Arg-Phe-Phe-Dap-Ala-NPhe-DPro]
(SEQ ID NO: 27)
c[Pro-Arg-Phe-Phe- [symbol] -Ala-Phe-DPro]
(SEQ ID NO: 28)
c[Pro-NArg-Phe-The-Asn-Ala-The-DPro]
(SEQ ID NO: 29)
c[Pro-Arg-NPhe-Phe-Asn-Ala-Phe-DPro]
(SEQ ID NO: 30)
c[Pro-Arg-Phe-Phe-Asn-NAla-Phe-DPro]
(SEQ ID NO: 31)
c[Pro-Arg-Phe-Phe-Asn-Ala-NPhe-DPro]

and salts thereof.

In one embodiment, the compound of invention is selected from the group consisting of:

(SEQ ID NO: 3)
c[Pro-Arg-Phe-Phe-Asn-Val-Phe-DPro]
(SEQ ID NO: 7)
c[Pro-Arg-Phe-Phe-Dap-Val-Phe-DPro]
(SEQ ID NO: 9)
c[Pro-Arg-Tyr-Phe-Dap-Ala-Phe-DPro]
(SEQ ID NO: 11)
c[Pro-Arg-Phe-NPhe-Asn-Ala-Phe-DPro]
(SEQ ID NO: 12)
c[Pro-Arg-Phe-NPhe-Dap-Ala-Phe-DPro]

and salts thereof.
In one embodiment, the compound of invention is c[Pro-Arg-Phe-Phe-Asn-Val-Phe-DPro] (SEQ ID NO:3), or a salt thereof.
In one embodiment, the compound of invention is c[Pro-Arg-Phe-Phe-Dap-Val-Phe-DPro] (SEQ ID NO:7), or a salt thereof.
In one embodiment, the compound of invention is c[Pro-Arg-Tyr-Phe-Dap-Ala-Phe-DPro] (SEQ ID NO:9), or a salt thereof.
In one embodiment, the compound of invention is c[Pro-Arg-Phe-NPhe-Asn-Ala-Phe-DPro] (SEQ ID NO:11), or a salt thereof.
In one embodiment, the compound of invention is c[Pro-Arg-Phe-NPhe-Dap-Ala-Phe-DPro] (SEQ ID NO:12), or a salt thereof.

In one embodiment, the compound of invention is a cyclic peptide or peptoid, comprising an amino acid sequence having at least 80% sequence identity to:

(SEQ ID NO: 13)
Pro-Arg-Phe-Phe-Asn-Val-Phe-DPro
(SEQ ID NO: 14)
Pro-His-Phe-Phe-Asn-Ala-The-DPro
(SEQ ID NO: 15)
Pro-Arg-Tyr-Phe-Asn-Ala-Phe-DPro
(SEQ ID NO: 16)
Pro-Cys-Phe-Phe-Asn-Ala-Phe-DPro
(SEQ ID NO: 17)
Pro-Arg-Phe-Phe-Dap-Val-Phe-DPro
(SEQ ID NO: 18)
Pro-His-Phe-Phe-Dap-Ala-Phe-DPro
(SEQ ID NO: 19)
Pro-Arg-Tyr-Phe-Dap-Ala-Phe-DPro
(SEQ ID NO: 20)
Pro-Cys-Phe-Phe-Dap-Ala-Phe-DPro
(SEQ ID NO: 21)
Pro-Arg-Phe-NPhe-Asn-Ala-Phe-DPro
(SEQ ID NO: 22)
Pro-Arg-Phe-NPhe-Dap-Ala-Phe-DPro
(SEQ ID NO: 32)
Pro-NArg-Phe-Phe-Dap-Ala-Phe-DPro
(SEQ ID NO: 33)
Pro-Arg-NPhe-Phe-Dap-Ala-Phe-DPro
(SEQ ID NO: 34)
Pro-Arg-Phe-Phe-Dap-NAla-Phe-DPro
(SEQ ID NO: 35)
Pro-Arg-Phe-The-Dap-Ala-NPhe-DPro
(SEQ ID NO: 36)
Pro-Arg-Phe-The- [symbol] -Ala-Phe-DPro
(SEQ ID NO: 37)
Pro-NArg-Phe-Phe-Asn-Ala-Phe-DPro
(SEQ ID NO: 38)
Pro-Arg-NPhe-Phe-Asn-Ala-Phe-DPro
(SEQ ID NO: 39)
Pro-Arg-Phe-Phe-Asn-NAla-The-DPro
or
(SEQ ID NO: 40)
Pro-Arg-Phe-Phe-Asn-Ala-NPhe-DPro.

In one embodiment, the compound of invention is a cyclic peptide, comprising an amino acid sequence having at least 80% sequence identity to:

(SEQ ID NO: 13)
Pro-Arg-Phe-Phe-Asn-Val-Phe-DPro
(SEQ ID NO: 14)
Pro-His-Phe-Phe-Asn-Ala-The-DPro
(SEQ ID NO: 15)
Pro-Arg-Tyr-Phe-Asn-Ala-Phe-DPro
(SEQ ID NO: 16)
Pro-Cys-Phe-Phe-Asn-Ala-Phe-DPro
(SEQ ID NO: 17)
Pro-Arg-Phe-Phe-Dap-Val-Phe-DPro
(SEQ ID NO: 18)
Pro-His-Phe-Phe-Dap-Ala-Phe-DPro
(SEQ ID NO: 19)
Pro-Arg-Tyr-Phe-Dap-Ala-Phe-DPro
(SEQ ID NO: 20)
Pro-Cys-Phe-Phe-Dap-Ala-Phe-DPro

In one embodiment, the compound of invention is a cyclic peptoid, comprising an amino acid sequence having at least 80% sequence identity to:

(SEQ ID NO: 21)
Pro-Arg-Phe-NPhe-Asn-Ala-The-DPro
(SEQ ID NO: 22)
Pro-Arg-Phe-NPhe-Dap-Ala-Phe-DPro
(SEQ ID NO: 32)
Pro-NArg-Phe-Phe-Dap-Ala-Phe-DPro
(SEQ ID NO: 33)
Pro-Arg-NPhe-Phe-Dap-Ala-Phe-DPro
(SEQ ID NO: 34)
Pro-Arg-Phe-Phe-Dap-NAla-Phe-DPro
(SEQ ID NO: 35)
Pro-Arg-Phe-Phe-Dap-Ala-NPhe-DPro
(SEQ ID NO: 36)
Pro-Arg-Pho-Phe- [symbol] -Ala-Phe-DPro
(SEQ ID NO: 37)
Pro-NArg-Phe-Phe-Asn-Ala-Phe-DPro
(SEQ ID NO: 38)
Pro-Arg-NPhe-Phe-Asn-Ala-Phe-DPro
(SEQ ID NO: 39)
Pro-Arg-Phe-Phe-Asn-NAla-Phe-DPro
or
(SEQ ID NO: 40)
Pro-Arg-Phe-Phe-Asn-Ala-NPhe-DPro.

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:32; SEQ ID NO:33; SEQ ID NO:34; SEQ ID NO:35; SEQ ID NO:36; SEQ ID NO:37; SEQ ID NO:38; SEQ ID NO:39; or SEQ ID NO:40.

In one embodiment, the compound of invention is a cyclic peptide or peptoid, comprising an amino acid sequence having at least 99% sequence identity to SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:32; SEQ ID NO:33; SEQ ID NO:34; SEQ ID NO:35; SEQ ID NO:36; SEQ ID NO:37; SEQ ID NO:38; SEQ ID NO:39 or SEQ ID NO:40.

In one embodiment, the compound of invention is a cyclic peptide or peptoid, comprising an amino acid sequence having at least 80% sequence identity to:

(SEQ ID NO: 3)
Pro-Arg-Phe-Phe-Asn-Val-Phe-DPro
(SEQ ID NO: 17)
Pro-Arg-Phe-Phe-Dap-Val-Phe-DPro
(SEQ ID NO: 19)
Pro-Arg-Tyr-Phe-Dap-Ala-Phe-DPro
(SEQ ID NO: 21)
Pro-Arg-Phe-NPhe-Asn-Ala-Phe-DPro
or
(SEQ ID NO: 22)
Pro-Arg-Phe-NPhe-Dap-Ala-Phe-DPro,

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:13, SEQ ID NO:17, SEQ ID NO:19, SEQ ID NO:21 or SEQ ID NO:22. In one embodiment, the compound of invention is a cyclic peptide or peptoid, consisting of an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:13, SEQ ID NO:17, SEQ ID NO:19, SEQ ID NO:21 or SEQ ID NO:22.

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:13. In one embodiment, the compound of invention is acyclic peptide or peptoid, consisting of an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:13. In certain embodiments, the cyclic peptide consists of SEQ ID NO:13.

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:17. In one embodiment, the compound of invention is a cyclic peptide or peptoid, consisting of an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:17. In certain embodiments, the cyclic peptide consists of SEQ ID NO:17.

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:19. In one embodiment, the compound of invention is a cyclic peptide or peptoid, consisting of an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:19. In certain embodiments, the cyclic peptide consists of SEQ ID NO:19.

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:21. In one embodiment, the compound of invention is a cyclic peptide or peptoid, consisting of an amino acid sequence having at least 81%, 82%, 83%, 84%, 85%, 86%, 87%, 88%, 89%, 90%, 91%, 92%, 93%, 94%, 95%, 9%%, 97%, 98%, 99% or 100/o sequence identity to SEQ ID NO:21. In certain embodiments, the cyclic peptide consists of SEQ ID NO:21.

In one embodiment, the compound of invention is a cyclic peptide or peptoid, comprising an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:22. In one embodiment, the compound of invention is a cyclic peptide or peptoid, consisting of an amino acid sequence having at least 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:22. In certain embodiments, the cyclic peptide consists of SEQ ID NO:22.

In certain embodiments, the cyclic peptide or peptoid is between about 5 to about 13 amino acids in length. In certain embodiments, the cyclic peptide or peptoid is between about 8 to about 13 amino acids in length. In certain embodiments, the cyclic peptide or peptoid is about 5, 6, 7, 8, 9, 10, 11, 12 or 13 amino acids in length. In certain embodiments, the cyclic peptide or peptoid is 8 amino acids in length.

In one embodiment, the compound of invention is a ligand for MC1R, MC3R, MC4R or MC5R. In one embodiment, the compound of invention is a ligand for MC4R or MC5R. In one embodiment, the compound of invention is a ligand for MC3R. In one embodiment, the compound of invention is a ligand for MC4R. In one embodiment, the compound of invention is a ligand for MC5R.

In one embodiment, the compound of invention binds to MC1R, MC3R, MC4R or MC5R. In one embodiment, the compound of invention selectively binds to MC1R, MC3R, MC4R or MC5R. For example, a compound of the invention may be at least 5, at least 10, at least 50, at least 100, at least 500, or at least 1,000 fold selective for a given melanocortin receptor (e.g., MC1R. MC3R. MC4R and/or MC5R) over another melanocortin receptor(s) in a selected assay (e.g., an assay described in the Examples herein).

In one embodiment, a compound of the invention described herein (e.g., SEQ ID NO:1-2, a compound of formula I, or salts thereof) is capable of modulating the activity or function of MC1R, MC3R, MC4R or MC5R.

In one embodiment, a compound of the invention is capable of modulating the activity or function of MC5R. In one embodiment, a compound of the invention is a MC5R antagonist.

In one embodiment, a compound of the invention is an AgRP-derived MC5R antagonist. In one embodiment, a compound of the invention is a MC5R inverse agonist.

In one embodiment, a compound of the invention is capable of modulating the activity or function of MC4R. In one embodiment, a compound of the invention is a MC4R antagonist. In one embodiment, a compound of the invention is a MC4R inverse agonist.

In one embodiment, a compound of the invention is capable of modulating the acitivity or function of MC3R. In one embodiment, a compound of the invention is a MC3R antagonist.

In one embodiment, a compound of the invention is an antagonist of MC3R, MC4R and MC5R.

One embodiment of the invention provides a composition (e.g., a pharmaceutical composition) comprising a compound described herein or compound of formula I, or a pharmaceutically acceptable salt thereof, and a pharmaceutically acceptable carrier.

One embodiment of the invention provides a dietary supplement comprising a compound described herein or compound of formula I, or a salt thereof.

Another embodiment of the invention provides a prodrug of a compound of formula I or a salt thereof. As used herein the term “prodrug” refers to a biologically inactive compound that can be metabolized in the bod to produce a biologically active form of the compound.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound of formula I herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 99% the absolute stereoisomer depicted.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Certain Methods of the Invention

Certain embodiments provide a method of modulating (e.g., increasing or decreasing) the activity/function of a melanocortin receptor in vitro or in vivo comprising contacting the receptor with an effective amount of a compound described herein, such as a compound of formula I, or a pharmaceutically acceptable salt thereof. In certain embodiments, the receptor is contacted in vitro. In certain embodiments, the receptor is contacted in vivo. In certain embodiments, the method is used to characterize a melaocortin receptor described herein, such as MC5R (e.g., characterize the physiological roles of MC5R). In certain embodiments, the method is used in medical therapy.

In certain embodiments, such a method comprises contacting a cell comprising the melanocortin receptor. In certain embodiments, the cell is in a mammal. In certain embodiments, the cell is contacted by administering a compound described herein, such as a compound of formula (I) or a salt thereof (e.g., a pharmaceutically acceptable salt thereof) to the mammal. In certain embodiments, the compound increases the activity of the melanocortin receptor (e.g., as compared to a control). In certain embodiments, the compound decreases the activity of the melanocortin receptor (e.g., as compared to a control).

One embodiment of the invention provides a compound described herein, such as a compound of formula I, or a pharmaceutically acceptable salt thereof, for use in modulating (e.g., increasing or decreasing) the activity/function of a melanocortin receptor in vitro or in vivo.

One embodiment of the invention provides the use of a compound described herein, such as a compound of formula I, or a pharmaceutically acceptable salt thereof for the manufacture of a medicament for modulating (e.g., increasing or decreasing) the activity/function of a melanocortin receptor in vitro or in vivo.

In one embodiment, the melanocortin receptor is MC1R.

In one embodiment, the melanocortin receptor is MC3R.

In one embodiment, the melanocortin receptor is MC4R.

In one embodiment, the melanocortin receptor is MC5R. Thus, in one embodiment, the activity/function of MC5R is modulated. In certain embodiments, the activity/function of MC5R is decreased (i.e., the compound is an antagonist and/or an inverse agonist of MC5R). For example, in certain embodiments, the compound described herein for antagonizing MC5R and/or as an inverse agonist of MC5R is c[Pro-Arg-Phe-Phe-Asn-Ala-Phe-DPro] (SEQ ID NO:1), c[Pro-Arg-Phe-Phe-Dap-Ala-Phe-DPro] (SEQ ID NO:2), c[Pro-Arg-Phe-Phe-Asn-Val-Phe-DPro] (SEQ ID NO:3), c[Pro-Arg-Phe-Phe-Dap-Val-Phe-DPro] (SEQ ID NO:7), c[Pro-Arg-Tyr-Phe-Dap-Ala-Phe-DPro] (SEQ ID NO:9), c[Pro-Arg-Phe-NPhe-Asn-Ala-Phe-DPro](SEQ ID NO:11), c[Pro-Arg-Phe-NPhe-Dap-Ala-Phe-DPro] (SEQ ID NO:12) or a salt thereof. In certain embodiments, the compound described herein for antagonizing MC5R is cPro-Arg-Phe-Phe-Asn-Val-Phe-DPro (SEQ ID NO:3), c[Pro-Arg-Phe-Phe-Dap-Val-Phe-DPro] (SEQ ID NO:7), c[Pro-Arg-Tyr-Phe-Dap-Ala-Phe-DPro] (SEQ ID NO:9), c[Pro-Arg-Phe-NPhe-Asn-Ala-Phe-DPro] (SEQ ID NO: 11), c[Pro-Arg-Phe-NPhe-Dap-Ala-Phe-DPro] (SEQ ID NO:12), or a salt thereof.

Thus, certain embodiments of the invention also provide a method of antagonizing MCR5 in an animal in need thereof, comprising administering to the animal an effective amount of a compound described herein, such as a compound of formula I, or a pharmaceutically acceptable salt thereof (e.g., SEQ ID NO:1, SEQ ID NO:2, SEQ ID NO:3, SEQ ID NO:7, SEQ ID NO:9, SEQ ID NO:11, or SEQ ID NO:12).

Certain embodiments also provide a compound described herein (e.g., a compound of formula I), or a pharmaceutically acceptable salt thereof, for antagonizing MCR5 in an animal in need thereof.

Certain embodiments provide the use of compound as described herein (e.g., a compound of formula I), or a pharmaceutically acceptable salt thereof, to prepare a medicament for antagonizing MCR5 in an animal in need thereof.

The invention also provides a method for treating a disease or disorder associated with MC5R in an animal (e.g., a mammal, such as a human) in need thereof comprising administering to the animal a compound as described herein, or a pharmaceutically acceptable salt thereof (e.g., a compound of formula I, such as SEQ ID NO:1, SEQ ID NO:2, SEQ ID NO:3, SEQ ID NO:7, SEQ ID NO:9, SEQ ID NO:11, or SEQ ID NO:12). The invention also provides a method for treating a disease or disorder associated with excessive activity of MC5R or overexpression of MC5R in an animal (e.g., a mammal, such as a human) comprising administering to the animal a compound as described herein, or a pharmaceutically acceptable salt thereof (e.g., a compound of formula I, such as SEQ ID NO:1, SEQ ID NO:2, SEQ ID NO:3, SEQ ID NO:7, SEQ ID NO:9, SEQ ID NO:11, or SEQ ID NO:12). In certain embodiments, the activity of MC5R is modulated following the administration of the compound. In certain embodiments, the activity of MC5R is antagonized following the administration of the compound.

The invention also provides a compound described herein, or a pharmaceutically acceptable salt thereof, for the prophylactic or therapeutic treatment of a disease or disorder associated with MC5R. The invention also provides a compound described herein, or a pharmaceutically acceptable salt thereof, for the prophylactic or therapeutic treatment of a disease or disorder associated with excessive activity of MC5R or overexpression of MC5R (e.g., as compared to a control).

Certain embodiments also provide the use of a compound herein, or a pharmaceutically acceptable salt thereof, to prepare a medicament for treating a disease or disorder associated with MC5R activity. Certain embodiments also provide the use of a compound herein, or a pharmaceutically acceptable salt thereof, to prepare a medicament for treating a disease or disorder associated with excessive activity of MC5R or overexpression of MC5R (e.g., as compared to a control).

Diseases and disorders associated with MC5R activity are known in the art and include, but are not limited to, exocrine gland disorder (e.g., lacrimal gland or sebaceous gland), acne, rosacea or sebaceous gland pathologies (e.g., lipid overproduction from sebaceous gland), metabolic disorders (e.g., obesity, cachexia or thermodysregulation, etc.), negative energy imbalance (e.g., cachexia or anorexia, etc), wasting disease (e.g., cachexia), mental disorders (e.g., anxiety, panic disorder, social phobia, depression, bipolar disorder, acute stress disorder or post-traumatic stress disorder), inflammatory conditions (e.g., overproduction of cytokines, such as interleukin 6) and cardiac hypertrophy (e.g., high glucose induced cardiac hypertrophy) (see also. US 2019/0255142 and Bednarek et al. J. Med. Chem. 2007, 50, 2530-2526, which are incorporated by reference herein).

Thus, in one embodiment, the disease or disorder associated with MC5R is exocrine gland disorder (e.g., lacrimal gland or sebaceous gland). In one embodiment, the disease or disorder associated with MC5R is acne, rosacea or sebaceous gland pathologies (e.g., lipid overproduction from sebaceous gland). In one embodiment, the disease or disorder associated with MC5R is a metabolic disorder (e.g., obesity, cachexia or thermodysregulation, etc.). In one embodiment, the disease or disorder associated with MC5R is a disease of negative energy imbalance (e.g., cachexia or anorexia, etc.). In one embodiment, the disease or disorder associated with MC5R is a wasting disease (e.g., cachexia). In one embodiment, the disease or disorder associated with MC5R is a mental disorder (e.g., anxiety, panic disorder, social phobia, depression, bipolar disorder, acute stress disorder or post-traumatic stress disorder). In one embodiment, the disease or disorder associated with MC5R is an inflammatory condition (e.g., overproduction of cytokines, such as interleukin 6). In one embodiment, the disease or disorder associated with MC5R is cardiac hypertrophy (e.g., high glucose induced cardiac hypertrophy).

Another embodiment of the invention provides a method of modulating (e.g., increasing or decreasing) the release of stress hormones in an animal in need thereof, comprising administering to the animal an effective amount of a compound described herein, such as a compound of formula I, or a pharmaceutically acceptable salt thereof.

Another embodiment of the invention provides a compound described herein, or a pharmaceutically acceptable salt thereof, for use in modulating (e.g., increasing or decreasing) the release of stress hormones in an animal in need thereof.

Another embodiment of the invention provides the use of a compound described herein, or a pharmaceutically acceptable salt thereof, for the manufacture of a medicament for modulating (e.g., increasing or decreasing) the release of stress hormones an animal in need thereof.

Another embodiment of the invention provides a method of modulating (e.g., increasing or decreasing) metabolic activity in an animal in need thereof, comprising administering to the animal an effective amount of a compound described herein, such as a compound of formula I, or a pharmaceutically acceptable salt thereof.

Another embodiment of the invention provides a compound described herein, or a pharmaceutically acceptable salt thereof, for use in modulating (e.g., increasing or decreasing) metabolic activity in an animal in need thereof.

Another embodiment of the invention provides the use of a compound described herein, or a pharmaceutically acceptable salt thereof, for the manufacture of a medicament for modulating (e.g., increasing or decreasing) metabolic activity in an animal in need thereof.

Another embodiment of the invention provides a method of modulating (e.g., increasing or decreasing) appetite in an animal in need thereof, comprising administering to the animal an effective amount of a compound described herein, such as a compound of formula I, or a pharmaceutically acceptable salt thereof.

Another embodiment of the invention provides a compound described herein, or a pharmaceutically acceptable salt thereof, for use in modulating (e.g., increasing or decreasing) appetite in an animal in need thereof.

Another embodiment of the invention provides the use of a compound described herein, or a pharmaceutically acceptable salt thereof, for the manufacture of a medicament for modulating (e.g., increasing or decreasing) appetite in an animal in need thereof.

Another embodiment of the invention provides a method of treating obesity or a disease associated with obesity in an animal in need thereof, comprising administering to the animal an effective amount of a compound described herein, such as a compound of formula I, or a pharmaceutically acceptable salt thereof.

The invention also provides a compound described herein, or a pharmaceutically acceptable salt thereof, for the prophylactic or therapeutic treatment of obesity or a disease associated with obesity.

The invention also provides the use of a compound described herein, or a pharmaceutically acceptable salt thereof, to prepare a medicament for treating obesity or a disease associated with obesity.

In one embodiment, the disease associated with MC5R is acne, rosacea or sebaceous gland pathology.

The invention also provides a method for treating acne, rosacea or sebaceous gland pathology in an animal (e.g., a mammal, such as a human) comprising administering to the animal a compound described herein, such as a compound of formula I, or a pharmaceutically acceptable salt thereof.

The invention also provides a compound described herein, or a pharmaceutically acceptable salt thereof, for the prophylactic or therapeutic treatment of acne, rosacea or sebaceous gland pathology.

The invention also provides the use of a compound described herein, or a pharmaceutically acceptable salt thereof, to prepare a medicament for treating acne, rosacea or sebaceous gland pathology.

In one embodiment, the disease associated with obesity is diabetes, cardiovascular disease or hypertension.

The invention also provides a method for treating cachaxia or a disease associated with cachaxia in an animal (e.g., a mammal, such as a human) comprising administering to the animal a compound described herein, such as a compound of formula I, or a pharmaceutically acceptable salt thereof.

The invention also provides a compound described herein, or a pharmaceutically acceptable salt thereof, for the prophylactic or therapeutic treatment of cachaxia or a disease associated with cachaxia.

The invention also provides the use of a compound described herein, or a pharmaceutically acceptable salt thereof, to prepare a medicament for treating cachaxia or a disease associated with cachaxia.

In one embodiment, the disease associated with cachaxia is cancer, congestive heart failure or chronic kidney disease.

A compound described herein, or a pharmaceutically acceptable salt thereof, may also be used for stimulating hunger, or increasing appetite, food intake or feeding behavior.

The ability of a compound described herein to, e.g., modulate appetite, modulate metabolic activity, to treat cachexia or a disease associated with cachexia, or to treat obesity or diseases associated with obesity (e.g., diabetes, cardiovascular disease or hypertension) may be tested using an assay known in the art or described in the Examples.

In certain embodiments, the compound is selected from the group consisting of: c[Pro-Arg-Phe-Phe-Asn-Ala-Phe-DPro] (SEQ ID NO:1), c[Pro-Arg-Phe-Phe-Dap-Ala-Phe-DPro](SEQ ID NO:2), c[Pro-Arg-Phe-Phe-Asn-Val-Phe-DPro] (SEQ ID NO:3); c[Pro-His-Phe-Phe-Asn-Ala-Phe-DPro] (SEQ ID NO:4); c[Pro-Arg-Tyr-Phe-Asn-Ala-Phe-DPro] (SEQ ID NO:5); c[Pro-Cys-Phe-Phe-Asn-Ala-Phe-DPro] (SEQ ID NO:6); c[Pro-Arg-Phe-Phe-Dap-Val-Phe-DPro] (SEQ ID NO:7); c[Pro-His-Phe-Phe-Dap-Ala-Phe-DPro] (SEQ ID NO:8); c[Pro-Arg-Tyr-Phe-Dap-Ala-Phe-DPro] (SEQ ID NO:9); c[Pro-Cys-Phe-Phe-Dap-Ala-Phe-DPro] (SEQ ID NO:10); c[Pro-Arg-Phe-NPhe-Asn-Ala-Phe-DPro] (SEQ ID NO:11); c[Pro-Arg-Phe-NPhe-Dap-Ala-Phe-DPro] (SEQ ID NO:12), c[Pro-NArg-Phe-Phe-Dap-Ala-Phe-DPro] (SEQ ID NO:23); c[Pro-Arg-NPhe-Phe-Dap-Ala-Phe-DPro] (SEQ ID NO:24); c[Pro-Arg-Phe-Phe-Dap-NAla-Phe-DPro] (SEQ ID NO:25); c[Pro-Arg-Phe-Phe-Dap-Ala-NPhe-DPro] (SEQ ID NO:26); c[Pro-Arg-Phe-Phe-NDab-Ala-Phe-DPro] (SEQ ID NO:27); c[Pro-NArg-Phe-Phe-Asn-Ala-Phe-DPro] (SEQ ID NO:28); c[Pro-Arg-NPhe-Phe-Asn-Ala-Phe-DPro] (SEQ ID NO:29); c[Pro-Arg-Phe-Phe-Asn-NAla-Phe-DPro] (SEQ ID NO:30); c[Pro-Arg-Phe-Phe-Asn-Ala-NPhe-DPro] (SEQ ID NO:31), and salts thereof.

In certain embodiments, the compound is selected from the group consisting of: c[Pro-Arg-Phe-Phe-Asn-Ala-Phe-DPro] (SEQ ID NO:1), c[Pro-Arg-Phe-Phe-Dap-Ala-Phe-DPro](SEQ ID NO:2), c[Pro-Arg-Phe-Phe-Asn-Val-Phe-DPro] (SEQ ID NO:3); c[Pro-His-Phe-Phe-Asn-Ala-Phe-DPro] (SEQ ID NO:4); c[Pro-Arg-Tyr-Phe-Asn-Ala-Phe-DPro] (SEQ ID NO:5); c[Pro-Cys-Phe-Phe-Asn-Ala-Phe-DPro] (SEQ ID NO:6); c[Pro-Arg-Phe-Phe-Dap-Val-Phe-DPro] (SEQ ID NO:7); c[Pro-His-Phe-Phe-Dap-Ala-Phe-DPro] (SEQ ID NO:8); c[Pro-Arg-Tyr-Phe-Dap-Ala-Phe-DPro](SEQ ID NO:9); c[Pro-Cys-Phe-Phe-Dap-Ala-Phe-DPro] (SEQ ID NO:10); c[Pro-Arg-Phe-NPhe-Asn-Ala-Phe-DPro] (SEQ ID NO:11); c[Pro-Arg-Phe-NPhe-Dap-Ala-Phe-DPro] (SEQ ID NO:12), and salts thereof.

In certain embodiments, the compound is a MC5R antagonist. For example, in certain embodiments, the compound is selected from the group consisting of: c[Pro-Arg-Phe-Phe-Asn-Ala-Phe-DPro] (SEQ ID NO:1), c[Pro-Arg-Phe-Phe-Dap-Ala-Phe-DPro] (SEQ ID NO:2), c[Pro-Arg-Phe-Phe-Asn-Val-Phe-DPro] (SEQ ID NO:3); c[Pro-Arg-Phe-Phe-Dap-Val-Phe-DPro] (SEQ ID NO:7); c[Pro-Arg-Tyr-Phe-Dap-Ala-Phe-DPro] (SEQ ID NO:9); c[Pro-Arg-Phe-NPhe-Asn-Ala-Phe-DPro] (SEQ ID NO:11); c[Pro-Arg-Phe-NPhe-Dap-Aa-Phe-DPro] (SEQ ID NO:12), and salts thereof. In certain embodiments, the compound is selected from the group consisting of: c[Pro-Arg-Phe-Phe-Asn-Val-Phe-DPro] (SEQ ID NO:3); c[Pro-Arg-Phe-Phe-Dap-Val-Phe-DPro] (SEQ ID NO:7); c[Pro-Arg-Tyr-Phe-Dap-Ala-Phe-DPro] (SEQ ID NO:9); c[Pro-Arg-Phe-NPhe-Asn-Ala-Phe-DPro] (SEQ ID NO:11); c[Pro-Arg-Phe-NPhe-Dap-Ala-Phe-DPro] (SEQ ID NO:12), and salts thereof. In certain embodiments, the compound is c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2), or a salt thereof.

In certain embodiments, the compound is c[Pro-Arg-Phe-Phe-Asn-Ala-Phe-DPro] (SEQ ID NO:1), or a salt thereof. In certain embodiments, the compound is c[Pro-Arg-Phe-Phe-Dap-Ala-Phe-DPro] (SEQ ID NO:2), or a salt thereof. In certain embodiments, the compound is c[Pro-Arg-Phe-Phe-Asn-Val-Phe-DPro] (SEQ ID NO:3), or a salt thereof. In certain embodiments, the compound is c[Pro-Arg-Phe-Phe-Dap-Val-Phe-DPro] (SEQ ID NO:7), or a salt thereof. In certain embodiments, the compound is c[Pro-Arg-Tyr-Phe-Dap-Ala-Phe-DPro](SEQ ID NO:9), or a salt thereof. In certain embodiments, the compound is c[Pro-Arg-Phe-NPhe-Asn-Ala-Phe-DPro] (SEQ ID NO:11), or a salt thereof. In certain embodiments, the compound is c[Pro-Arg-Phe-NPhe-Dap-Ala-Phe-DPro] (SEQ ID NO:12), or a salt thereof.

In certain embodiments, the compound is not c[Pro-Arg-Phe-Phe-Asn-Ala-Phe-DPro](SEQ ID NO:1). In certain embodiments, the compound is not c[Pro-Arg-Phe-Phe-Dap-Ala-Phe-DPro] (SEQ ID NO:2).

In certain embodiments, the animal is a mammal. In certain embodiments, the mammal is a human.

### Administration

Compounds described herein (e.g., compounds of formula I, including salts and prodrugs thereof) can be formulated as pharmaceutical compositions and administered to a mammalian host, such as a human patient in a variety of forms adapted to the chosen route of administration, i.e., orally or parenterally, by intravenous, intramuscular, intrathecal, topical, nasal, inhalation, suppository, sub dermal osmotic pump, intradermal or subcutaneous routes.

Thus, the present compounds may be systemically administered, e.g., orally, in combination with a pharmaceutically acceptable vehicle such as an inert diluent or an assimilable edible carrier. They may be provided as lyophilized formulation (e.g., with trehalose or sucrose as cryo-lyoprotectant, and/or mannitol as bulking agent), or enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously, intrathecally or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds described herein to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of the invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents. For example, compounds of the invention (e.g., compounds of formula (I)), or salts thereof, may be administered with other agents that are useful for modulating appetite (i.e., increasing or decreasing), modulating metabolic activity or treating a disease or disorder associated with MC5R. Accordingly, in one embodiment the invention also provides a composition comprising a compound described herein (e.g., compound of formula (I)), or a pharmaceutically acceptable salt thereof, at least one other therapeutic agent, and a pharmaceutically acceptable diluent or carrier. The invention also provides a kit comprising a compound described herein (e.g, a compound of formula (I)), or a pharmaceutically acceptable salt thereof, at least one other therapeutic agent, packaging material, and instructions for administering a compound described herein (e.g., a compound of formula (I)) or the pharmaceutically acceptable salt thereof and the other therapeutic agent or agents to an animal to modulating appetite (i.e., increasing or decreasing), modulating metabolic activity or treating a disease or disorder associated with MC5R.

### Certain Definitions

The term “alkyl”, by itself or as part of another substituent, means, unless otherwise stated, a straight or branched chain hydrocarbon radical, having the number of carbon atoms designated (i.e., C₁₋₄ means one to four carbons). Non limiting examples of “alkyl” include methyl, ethyl, propyl, isopropyl, butyl, iso-butyl, sec-butyl.

The term “halo” means fluoro, chloro, bromo, or iodo.

The term “haloalkyl” means an alkyl that is optionally substituted with one or more (e.g., 1, 2, 3, 4, or 5) halo. Non limiting examples of “haloalkyl” include iodomethyl, bromomethyl, chloromethyl, fluoromethyl, trifluoromethyl, 2-chloroethyl, 2-fluoroethyl, 2,2,2-trifluoroethyl 2,2-difluoroethyl and pentafluoroethyl.

The term “amino acid,” comprises the residues of the natural amino acids (e.g. Ala, Arg, Asn, Asp, Cys, Glu, Gln, Gly, His, Hyl, Hyp, Ile, Leu, Lys. Met, Phe, Pro, Ser. Thr, Trp, Tyr, and Val) in D or L form, as well as unnatural amino acids (e.g. Dap, PyrAla, ThiAla, (pCl)Phe, (pNO₂)Phe, ε-Aminocaproic acid, Met[O₂], dehydPro, (3I)Tyr, norleucine (Nle), para-1-phenylalanine ((pI)Phe), 2-napthylalanine (2-Nal), β-cyclohexylalanine (Cha), β-alanine (β-Ala), phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid (Tic), penicillamine, omithine, citruline, α-methyl-alanine, para-benzoylphenylalanine, phenylglycine, propargylglycine, sarcosine, and tert-butylglycine) in D or L form. The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the anuno terminus, or through any other convenient point of attachment, such as, for example, through the sulfur of cysteine. An amino acid specifically recited herein refers to its L-form, unless specified otherwise.

The term “peptide” describes a sequence of 2 to 25 amino acids (e.g. as defined hereinabove) or peptidyl residues. The sequence may be linear or cyclic. For example, a cyclic peptide can be prepared or may result from the formation of amide bonds or disulfide bridges between two cysteine residues in a sequence. When a peptide is cyclic, it can be illustrated as “c[peptide sequence]”. A peptide can be linked to the remainder of a compound of formula I through the carboxy terminus, the amino terminus, or through any other convenient point of attachment, such as, for example, through the sulfur of a cysteine. In certain embodiments, a peptide comprises 3 to 10, or 4 to 8 amino acids. In certain embodiments, a peptide comprises 5 to 13 amino acids, or 5 to 9 amino acids, or 8 to 13 amino acids or 8 to 9 amino acids. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right. The term “dipeptide” refers to a peptide comprising two amino acids joined through an amide bond. The term “tripeptide” means a peptide comprising three amino acids joined through two amide bonds. The terms “protein,” “peptide” and “polypeptide” are used interchangeably herein.

The term “peptoid” describes a peptide comprising one or more amino acids that are substituted with a N-substituted glycine such as N-Benzylglycine (NPhe), N-(3-guanidino-propyl)-glycine (NArg), N-(2-aminoethyl)-glycine (NDab), N-methylglycine (NAla).

As used herein, the term “compound” includes peptides and cyclic peptides described herein (e.g., compounds of formula I).

As used herein, the term “residue of an amino acid” means a portion of an amino acid. For example, variables X¹, X², X³, X⁴, X⁵ and X⁶ may be residues of an amino acid, wherein certain atoms (e.g., H or OH) have been removed to link the amino acids via a peptide bond.

The following terms are used to describe the sequence relationships between two or more sequences (e.g., polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length peptide sequence or the complete peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4:11; the local homology algorithm of Smith et al. (1981) Adv. Appl. Math, 2:482; the homology alignment algorithm of Needleman and Wunsch, (1970) JMB, 48:443; the search-for-similarity-method of Pearson and Lipman, (1988) Proc. Natl. Acad. Sci. USA, 85:2444; the algorithm of Karlin and Altschul, (1990) Proc. Natl. Acad. Sci. USA, 87:2264, modified as in Karlin and Altschul, (1993) Proc. Nat. Acad. Sci. USA. 90:5873.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package. Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237; Higgins et al. (1989) CABIOS 5:151; Corpet et al. (1988) Nucl. Acids Res. 16:10881; Huang et al. (1992) CABIOS 8:155; and Pearson et al. (1994) Meth. Mol. Biol. 24:307. The ALIGN program is based on the algorithm of Myers and Miller, supra. The BLAST programs of Altschul et al. (1990) JMB, 215:403; Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25:3389. Alternatively. PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The terms “treat” and “treatment” refer to both therapeutic treatment and prophylactic or preventative measures, wherein the object is to prevent or decrease an undesired physiological change or disorder, such as a metabolic disorder (e.g., obesity) or a disease associated with the metabolic disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to, e.g., humans, higher non-human primates, rodents, domestic, cows, horses, pigs, sheep, dogs and cats. In one embodiment, the mammal is a human.

The invention will now be illustrated by the following non-limiting Examples.

### Example 1. Incorporation of Agouti-Related Protein (AgRP) Human Single Nucleotide Polymorphisms (SNPs) in the AGRP-Derived Macrocyclic Scaffold c[Pro-Arg-Phe-Phe-Asn-Ala-Phe-DPro] (SEQ ID NO: 1) Decreases Melanocortin-4 Receptor Antagonist Potency and Results in the Discovery of Melanocortin-S Receptor Antagonists

### Abstract:

While the melanocortin receptors are known to control a variety of physiological functions, the potential physiological roles of the melanocortin-5 receptor (MC5R) have not been clearly elucidated due to a lack of potent selective ligands. It is hypothesized that an exposed ß-hairpin loop composed of six residues (Arg-Phe-Phe-Asn-Ala-Phe (SEQ ID NO: 41)) is imperative for the endogenous AgRP, an MC3R antagonist and MC4R inverse agonist and antagonist, to bind and function. Within the active-loop region of AgRP, four human single nucleotide polymorphisms (SNPs) resulting in missense mutations were deposited in the NIH Variation Viewer database when viewed on Nov. 18, 2017. These SNPs, located at positions Arg111Cys, Arg111His, Phe112Tyr, and Ala115Val in the full-length AgRP amino acid sequence, which were incorporated into the macrocyclic scaffold c[Pro¹-Arg²-Phe³-Phe⁴-Xaa⁵-Ala⁶-Phe⁷-DPro⁸] (SEQ ID NO: 42), where Xaa was Dap⁵ or Asn⁵, to explore the potential functional effects of these SNPs in a simplified AgRP scaffold. All SNP containing peptides lowered potency in a cAMP accumulation assay at least 10-fold as compared to the parent sequences at the human and mouse MC4Rs. Unexpectantly, compounds MDE 6-82-3c, ZMK 2-82, MDE 6-82-1c, ZMK 2-85, and ZMK 2-112 were found to be MC5R antagonists with cAMP based pA₂ potencies ranging from 54 to 580 nM. This study demonstrated the usefulness of a truncated mimetic scaffold in identifying potentially modifying SNPs in larger signaling molecules like AgRP. Additionally, the first AgRP based chemotypes resulting in MC5R antagonists are reported, providing new molecular probes to characterize the physiological roles of the MC5R.

### Introduction

Five unique melanocortin receptors (MCRs) have been identified to date. (Chhajlani et al., FEBS Lett, 309 (3): 417-420, 1992; Mountjoy et al., Science, 257 (5074); 1248-1251, 1992; Chhajlani et al., Biochem Biophys Res Commun, 195 (2): 866-873, 1993; Gantz et al., J Biol Chem, 268 (11): 8246-8250, 1993; Gantz et al., J Biol Chem, 268 (20): 15174-15179, 1993; Roselli-Rehfuss et al., Proc Natl Acad Sci USA, 90 (19): 8856-8860, 1993; Gantz et al., Biochem Biophys Res Commun, 200 (3): 1214-1220, 1994; Griffon et al., Biochem Biophys Res Commun, 200 (2): 1007-1014, 1994) These receptors are members of the family A of G protein-coupled receptors (GPCRs). The MCRs have been shown to be involved in several different physiological functions such as skin pigmentation (MC1R), (Chhajlani et al., FEBS Lett, 309 (3): 417-420, 1992; Mountjoy et al., Science, 257 (5074): 1248-1251, 1992) steroidogenesis (MC2R), (Haynes et al., J Biol Chem, 225 (1): 115-124, 1957; Mountjoy et al., Science, 257 (5074): 1248-1251, 1992) and energy homeostasis (MC3/4R). (Huszar et al., Cell, 88 (1): 131-141, 1997; Butler et al., Endocrinology, 141 (9): 3518-3521, 2000; Chen et al., Nat Genet, 26 (1): 97-102, 2000; Irani et al., Eur J Pharmacol, 660 (1): 80-87, 2011) While many biological activities have been attributed to the MC1-4Rs, the physiological role(s) of the MC5R have not been fully identified and characterized for this most ubiquitously expressed melanocortin receptor. Based upon the phenotype of the MC5RKO mice, the MC5R has been reported to be involved in sebaceous gland function. (Chen et al., Cell, 91 (6): 789-798, 1997) The melanocortin receptor family is stimulated by several endogenous peptides including α-, ß-, and γ-melanocyte stimulating hormone (MSH) and adrenocorticotropin hormone (ACTH), all processed from the pro-opiomelanocortin (POMC) gene transcript. (Nakanishi et al., Nature. 278 (5703): 423-427, 1979) In addition to these agonists, there are two known naturally occurring antagonists for the melanocortin receptor family: agouti-signaling protein (ASP) (Bultman et al., Cell, 71 (7): 1195-1204, 1992; Miller et al., Genes Dev, 7 (3): 454-467, 1993) and agouti-related protein (AgRP). (Fong et al., Biochem Biophys Res Commun, 237 (3): 629-631, 1997; Ollmann et al., Science, 278 (5335): 135-138, 1997) These antagonist peptides both contain an Arg-Phe-Phe motif in the carboxyl terminal region, as reviewed, (Dinulescu et al., J Biol Chem, 275 (10): 6695-6698, 2000) which has been proposed to be the sequence responsible for receptor affinity and antagonist activity. (Kiefer et al., Biochemistry, 37 (4): 991-997, 1998; Tota et al., Biochemistry, 38 (3): 897-904, 1999) AgRP is expressed centrally in the arcuate nucleus of the hypothalamus (Ollmann et al., Science, 278 (5335): 135-138, 1997) and antagonizes the MC3R and MC4R, (Gantz et al., J Biol Chem, 268 (20): 15174-15179, 1993: Mountjoy et al., Mol Endocrinol, 8 (10): 1298-1308, 1994; Ollmann et al., Science, 278 (5335): 135-138, 1997) effectively increasing feeding behavior. (Fan et al., Nature, 385 (6612): 165-168, 1997; Huszar et al., Cell, 88 (1): 131-141, 1997; Giraudo et al., Brain Res, 809 (2): 302-306, 1998; Morton et al., Int J Obes Relat Metab Disord, 25 Suppl 5 S56-62, 2001) Additionally, AgRP functions as an inverse agonist at the MC4R, decreasing the amount of cyclic adenosine monophosphate (cAMP) produced by the cell in the absence of the agonist ligand stimulation (Haskell-Luevano et al., Regul Pept, 99 (1): 1-7, 2001; Nijenhuis et al., Mol Fndocrinol, 15 (1): 164-171, 2001) Feeding studies reported that central administration of melanocortin agonists such as NDP-MSH (a synthetic potent analogue of α-MSH) or melanotan II (MTII, a potent cyclic analogue of α-MSH) decreased food intake and prevented weight gain in mice. (Sawyer et al., Proc Natl Acad Sci USA, 77 (10): 5754-5758, 1980; Al-Obeidi et al., J Med Chem, 32 (12): 2555-2561, 1989; Chen et al., Transgenic Res, 9 (2): 145-154, 2000; Pierroz et al., Diabetes, 51 (5): 1337-1345, 2002; Adank et al., ACS Chem Neurosci, 9 (2):320-327, 2018) Contrarily, central injections of AgRP antagonist prompted long-acting increases in feeding behavior in fasted or sated animals. (Ollmann et al., Science, 278 (5335): 135-138, 1997 Hagan et al., Am J Physiol Regul Integr Comp Physiol, 279 (1): R47-52, 2000; Kim et al., Diabetes, 49 (2): 177-182, 2000; Wirth et al., Peptides, 21 (9): 1369-1375, 2000; Adank et al., ACS Chem Neurosci, 9 (2): 320-327, 2018)

In addition to the reported activity of AgRP at the MC3R and MC4R, there have been mixed reports about AgRP at the MC5R. Pooled fractions of recombinant AgRP using a baculovirus expression system in insect cells resulted in a rightward potency shift of α-MSH in HEK293 cells expressing the hMC5R at 100 nM concentrations. (Ollmann et al., Science, 278 (5335): 135-138, 1997) Another report using recombinant AGRP expressed from COS-7 cells indicated that AgRP was unable to displace radiolabeled NDP-MSH at the hMC5R expressed in L cells at up to 40 nM concentrations, although functional activity was not determined. (Fong et al., Biochem Biophys Res Commun, 237 (3); 629-631, 1997) A third report using chemically synthetized AgRP ligands [either the hAGRP(87-132)C-terminal domain, or a fusion of the mAGRP(21-85) to the hAGRP(87-132) generating the (Leu127Pro)AGRP] indicated that AgRP could functionally antagonize α-MSH at the MC5R at 100 nM concentrations, and could displace radiolabeled NDP-MSH (IC₅₀=310 nM) and radiolabeled AgRP (IC₅₀=26 nM) at the MC5R. (Yang et al., Mol Endocrinol, 13 (1): 148-155, 1999) The differing reported activities could be due to the activity ranges assayed, varying degrees of AgRP purity based upon the methods used to generate the peptide, or cellular assay conditions between the different reports. Previous reports of AgRP derived ligands have not reported MC5R antagonist activity, although inverse agonist efficacy has previously been observed. (Ericson et al., J Med Chem, 60 (19): 8103-8114, 2017; Fleming et al., ACS Chem Neurosci, 9 (5): 1141-1151, 2018)

Melanocortin ligands like MTII were originally developed before cloning of the receptors and to function as MCIR agonists in frog and lizard skin pigmentation assay. (Al-Obeidi et al., J Med Chem, 32 (12): 2555-2561, 1989; Alobeidi et al., J Am Chem Soc, 111 (9): 3413-3416, 1989) After cloning of the receptors, these compounds were developed for the clinic to prompt melanogenesis (tanning of the skin), aide in melanoma detection, and as melanoma chemotherapeutics. (Levine et al., J Invest Dermatol, 89 (3): 269-273, 1987; Levine et al., JAMA, 266 (19): 2730-2736, 1991; Lan et al., J Pharm Sci, 83 (8): 1081-1084, 1994; Hadley et al., Pigment Cell Res, 9 (5): 213-234, 1996) Since the early 2000's the MC4R, and to a lesser extent the MC3R, have been targeted due to their role in energy homeostasis and clinical indications in obesity and anorexia. (Butler et al., Trends Genet, 17 (10): S50-54, 2001; Marks et al., Cancer Res, 61 (4): 1432-1438, 2001; Vink et al., Mol Psychiatry, 6 (3): 325-328, 2001; Wisse et al., Endocrinology, 142 (8): 3292-3301, 2001; Lubrano-Berthelier et al., Diabetes, 52 (12): 2996-3000, 2003) Obesity has grown to be an epidemic, with projections estimating that over 50% of Americans will be overweight or obese by 2030. (Finkelstein et al., Am J Prev Med, 42 (6): 563-570, 2012) Approximately 5% of these cases are monogenic and linked to a disrupted central melanocortin system, where patients' obesity manifests in early childhood. (Vaisse et al., J Clin Invest, 106 (2): 253-262, 2000; Farooqi et al., NEngl J Med, 348 (12): 1085-1095, 2003; Yeo et al., Hum Mol Genet, 12 (5): 561-574, 2003) Diseases of negative energy imbalance, causing failure to thrive in young children, anorexia, and cachexia (disease-associated wasting) are also concerning because a decreased drive to eat can stunt growth in children and delay the body's response to healing. (Marks et al., Cancer Res, 61 (4): 1432-1438, 2001; Vink et al., Mol Psichiatry, 6 (3): 325-328, 2001; Wisse et al., Endocrinology, 142 (8): 3292-3301, 2001; Ge et al., Brain Res, 957 (1): 42-45, 2002; Weyermann et al., PLoS One, 4 (3): e4774, 2009) Manipulation of the melanocortin system in either direction could offer a viable solution to combating these diseases and their associated comorbidities.

Activation of the MC4R leads to a decrease in food intake, but compounds that have made it into the clinic have ultimately failed due to unwanted side effects like increases in blood pressure, (Greenfield et al., N Engl J Med, 360 (1): 44-52, 2009) darkening of the skin, (Kuhnen et al., N Engl J Med, 375 (3): 240-246, 2016; Clement et al., Nat Med 24 (5): 551-555, 2018) and increased erectile function, (Dorr et al., Life Sci, 58 (20): 1777-1784, 1996) as reviewed by Ericson et al. (Ericson et al., Biochim Biophs Acta Mol Basis Dis, 1863 (10 Pt A): 2414-2435, 2017) One compound, RM-493 (Setmelanotide), manufactured by Rhythm Pharmaceuticals, has seen clinical success in obese patients that do not produce the POMC gene transcript by decreasing body weight without the increase in blood pressure seen in the clinic with other melanocortin compounds. (Chen et al., J Clin Endocrinol Metab, 100 (4): 1639-1645, 2015) However, one patient with POMC deficiency was reported to have a decrease in diastolic blood pressure while being treated with RM-493 that was present during the extension phase of the study as well. (Kuhnen et al., N Engl J Med, 375 (3): 240-246, 2016) RM-493 has also been investigated in a Phase II trial in patients with rare genetic obesities (POMC deficiency, leptin/leptin receptor deficiency, Prader-Willi syndrome, Bardet-Biedl, etc.) clinical trial: NCT03013543. No drugs aimed towards increasing appetite through the melanocortin cascade have made it to the clinic to date.

Within the melanocortin system, many single nucleotide polymorphisms (SNPs) have been identified in the receptors, (Vaisse et al., Nat Genet, 20 (2): 113-114, 1998; Yeo et al., Nat Genet, 20 (2): 111-112, 1998; Vaisse et al., J Clin Invest, 106 (2): 253-262, 2000; Wong et al., Diabetes Res Clin Pract, 58 (1): 61-71, 2002; Feng et al., Diabetes, 54 (9): 2663-2667, 2005; Mencarelli et al., Eur J Hum Genet, 16 (5): 581-586, 2008; Santos et al., PLoS One, 6 (6): e19934, 2011) agonists. (Krude et al., Nat Genet, 19 (2): 155-157, 1998; Challis et al., Hum Mol Genet, 11 (17): 1997-2004, 2002) and antagonists, (Vink et al., Mol Psychiatry, 6 (3): 325-328, 2001; Argyropoulos et al., J Clin Endocrinol Metab, 87 (9): 4198-4202, 2002; Marks et al., Am J Med Genet A, 126A (3): 267-271, 2004; de Rijke et al., Biochem Pharmacol, 70 (2): 308-316, 2005; Bonilla et al., Int J Obes (Lond), 30 (4): 715-721, 2006; Kalnina et al., BMC Med Genet, 10 63, 2009) which may be linked to a predisposition for an obese or lean phenotype in humans. Receptor polymorphisms can alter protein conformation or structure thereby decreasing the affinity of the normally processed ligands. (Vaisse et al., Nat Genet, 20 (2): 113-114, 1998; Yeo et al., Nat Genet, 20 (2): 111-112, 1998; Vaisse et al., J Clin Invest, 106 (2): 253-262, 2000; Wong et al., Diabetes Res Clin Pract, 58 (1): 61-71, 2002; Feng et al., Diabetes, 54 (9): 2663-2667, 2005; Mencarelli et al., Eur J Hum Genet, 16 (5): 581-586, 2008) Mutations within genes that code for the peptide ligands can prevent the peptides from being synthesized or processed properly, alter ligand affinity or activity at the receptor, disrupting the natural signaling cascade and ultimately altering the phenotype of the patient. (Krude et al., Nat Genet, 19 (2): 155-157, 1998; Challis et al., Hum Mol Genet, 11 (17): 1997-2004, 2002; de Rijke et al., Biochem Pharmacol, 70 (2): 308-316, 2005; Bonilla et al., Int J Obes (Lond), 30 (4): 715-721, 2006; Ilnytska et al., Cell Mol Life Sci, 65 (17): 2721-2731, 2008; Kalnina et al., BMC Med Genet, 10 63, 2009; Li et al., Int J Obes (Lond), 38 (5); 724-729, 2014)

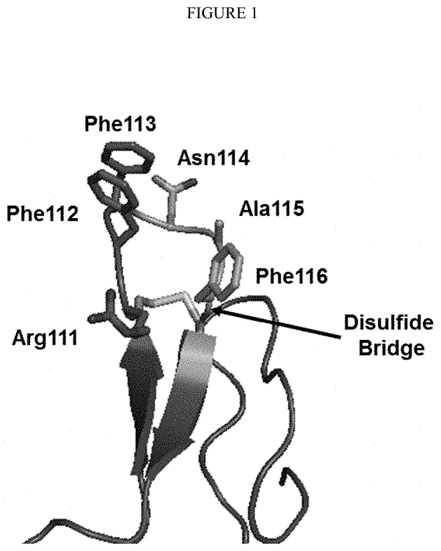

This study focused on exploring four single nucleotide polymorphisms that result in missense mutations in the putative active loop of AgRP, containing the Arg-Phe-Phe amino acid motif and the three following residues Asn, Ala, and Phe. This sequence is located on an exposed ß-hairpin loop in the structure of AgRP (FIG. 1). (Bolin et al., FEBS Lett, 451 (2): 125-131, 1999; Millhauser et al., Ann N Y Acad Sci, 994 27-35, 2003) Truncation studies have demonstrated that the cysteine-rich carboxyl terminus of AgRP is sufficient to modulate receptor activity at the MC3R and MC4R, and possibly the MC5R as discussed above. (Ollmann et al., Science, 278 (5335): 135-138, 1997; Quillan et al., FEBS Lett, 428 (1-2): 59-62, 1998; Millhauser et al., Ann N Y Acad Sci, 994 27-35, 2003) In the native sequence the loop is exposed between a disulfide bridge, FIG. 1.

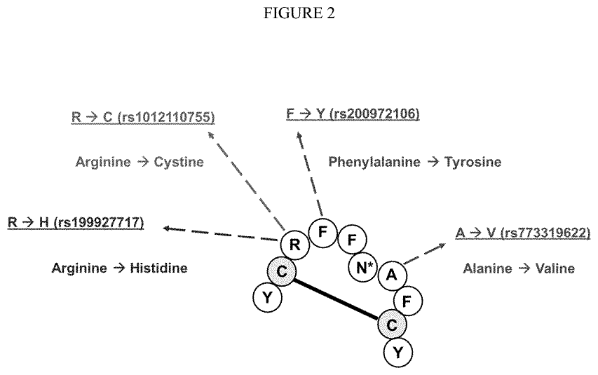

The AgRP antagonist, in its native form, is 132 amino acids in length, with five disulfide bonds throughout the carboxyl terminus: however, biologically active AgRP is attributed to residues 87-132. (Ollmann et al., Science, 278 (5335): 135-138, 1997: Yang et al., Mol Endocrinol, 13 (1): 148-155, 1999) Replicating this protein in its entirety or in its active form, to study multiple polymorphisms is costly and labor-intensive, therefore driving the discovery of smaller, more synthetically amenable probes and scaffolds. In 2015, we reported the discovery that the macrocyclic octapeptide AgRP mimetic scaffold, c[Pro-Arg-Phe-Phe-Asn-Ala-Phe-DPro] (SEQ ID NO: 1), was reported to have nanomolar potency at the MC4R. (Ericson et al., J Med Chem, 58 (11): 4638-4647, 2015) This scaffold uses a DPro-Pro head-to-tail cyclization to create a macrocycle ligand postulated to orient the key pharmacophore AGRP based side-chain groups in a similar conformation to that of the native AGRP endogenous antagonist. This macrocycle is smaller by 37 amino acids from AgRP (87-132) and retains nanomolar MC4R antagonist potency. This macrocycle is amenable to substitutions, allowing for the investigation of multiple potentially deleterious mutations within the putative AGRP active loop. The SNPs were accessed from the NIH Variation Viewer in November 2017 (ncbi.nlm.nih.gov/variation/view/). The mutations are R111H (rs199927717). R111C (rs1012110755), F112Y (rs200972106), and A115V (rs773319622) based upon the full length AGRP amino acid sequence, FIG. 2. (Ericson et al., ACS Chem Neurosci, 9 (6): 1235-1246, 2018) These mutations were systematically incorporated and assayed in the macrocyclic octapeptide scaffold. The polymorphisms were also incorporated into the scaffold with the endogenous Asn to diaminopropionic acid (Dap) substitution, as this modification was previously reported to be equipotent to AgRP(87-132) at the MC4R. (Ericson et al., J Med Chem, 58 (11): 4638-4647, 2015) Screening deposited mutations from a publicly available database versus using the genome sequenced from a small subset of an afflicted population, which has previously been the source of polymorphisms, (Krude et al., Nat Genet, 19 (2): 155-157, 1998; Vaisse et al., Nat Genet, 20 (2): 113-114, 1998; Vaisse et al., J Clin Invest, 106 (2): 253-262, 2000; Kuhnen et al., N Engl J Med, 375 (3): 240-246, 2016; Clement et al. Nat Med, 24 (5): 551-555, 2018; Challis et al., Hum Mol Genet, 11 (17): 1997-2004, 2002) allows for a broader understanding of the melanocortinergic control of energy homeostasis through the central receptors, and potentially other physiologically relevant functions through the peripheral receptors. In addition, this broad sourcing allows for a greater understanding of the contribution of polymorphisms in monogenic diseases. To fully characterize these SNPs, they would need to be synthesized in the full length of AgRP. It is hypothesized that the octapeptide scaffold can be utilized as a novel tool to screen polymorphisms that may be disruptive to AgRP signaling at the MCRs and provide new chemical probes to investigate MCR physiology in vivo. Using the two previously mentioned scaffolds, an eight-compound library was assayed for activity at the mouse MC1R, MC3R, MC4R, and MC5R and the human MC4R. Compounds were not tested at the MC2R as it has previously only been activated by full-length ACTH. (Haynes et al., J Biol Chem, 225 (1): 115-124, 1957; Mountjoy et al., Science, 257 (5074): 1248-1251, 1992)

### Results:

All peptides were synthesized using a microwave-assisted synthesizer and standard fluorenylmethoxycarbonyl (Fmoc) techniques. (Carpino et al., J Am Chem Soc, 92 (19): 5748, 1970: Carpino et al., Journal of Organic Chemistry. 37 (22): 3404, 1972) Peptides were cleaved from the resin and cyclized using BOP and HOBt, with their side chain protecting groups intact as previously described. (Ericson et al., J Med Chem, 58 (11): 4638-4647, 2015; Ericson et al., J Med Chem, 60 (19); 8103-8114, 2017; Ericson et al., ACS Chem Neurosci, 9 (12): 3015-3023, 2018; Fleming et al., ACS Chem Neurosci, 9 (5): 1141-1151, 2018) Peptides were purified to >95% purity using semi-preparative reverse-phase high performance liquid chromatography (RP-HPLC) after removing the protecting groups from the residues. Purity was determined using an analytical RP-HPLC, using two distinct solvent systems, Table 1. Molecular weights were assessed using either MALDI-TOF/TOF or ESI-TOF/MS (University of Minnesota Mass Spectrometry Laboratory), Table 1. The potencies of these compounds in live cell cAMP accumulation assay are reported in Tables 2 and 3. The binding affinities in live cell ¹²⁵I-radiolabeled NDP-MSH binding assay are reported in Tables 4 and 5.

TABLE 1
Analytical data for peptides synthesized in this study.

| | | Retention Time | | | mass spectral | |
|---|---|---|---|---|---|---|
| | | (min)^{a} | | M | analysis | Purity |
| Peptide | Sequence | System 1 | System 2 | (calc) | (M + 1) | % |
| ZMK 2-82 | c[Pro-Arg-Phe-Phe-Asn-Val-Phe-DPro] | 19.5 | 31.1 | 1004.5 | 1005.6 | >98% |
| | SEQ ID NO: 3 | | | | | |
| ZMK 2-85 | c[Pro-Arg-Phe-Phe-Dap-Val-Phe-DPro] | 19.7 | 31.3 | 976.5 | 977.6 | >95% |
| | SEQ ID NO: 7 | | | | | |
| ZMK 2-96 | c[Pro-His-Phe-Phe-Asn-Ala-Phe-DPro] | 17.8 | 28.2 | 957.4 | 958.4 | >98% |
| | SEQ ID NO: 4 | | | | | |
| ZMK 2-99 | c[Pro-His-Phe-Phe-Dap-Ala-Phe-DPro] | 18.0 | 29.7 | 929.5 | 930.4 | >95% |
| | SEQ ID NO: 8 | | | | | |
| ZMK 2-110 | c[Pro-Arg-Tyr-Phe-Asn-Ala-Phe-DPro] | 15.4 | 24.6 | 992.5 | 993.5 | >99% |
| | SEQ ID NO: 5 | | | | | |
| ZMK 2-112 | c[Pro-Arg-Tyr-Phe-Dap-Ala-Phe-DPro] | 15.4 | 25.6 | 964.5 | 965.5 | >98% |
| | SEQ ID NO: 9 | | | | | |
| ZMK 3-18 | c[Pro-Cys-Phe-Phe-Asn-Ala-Phe-DPro] | 20.9 | 31.8 | 924.1 | 924.8 | >97% |
| | SEQ ID NO: 6 | | | | | |
| ZMK 3-20 | c[Pro-Cys-Phe-Phe-Dap-Ala-Phe-DPro] | 21.9 | 32.8 | 896.14 | 896.8 | >95% |
| | SEQ ID NO: 10 | | | | | |

^{a}Peptide retentioni times (min) are reported for solvent system 1 (10% acetonitrile in 0.1% trifluoroacetic
acid/water and a gradient to 90% acetonitrile over 35 min) and solvent system 2 (10% methanol in 0.1%
trifluoroacetic acid/water and a gradient to 90% methanol over 35 min). An analytical Vydac C18 column
(Vydac 218PT104) was used with a flow rate of 1.5 mL/min. The peptide purity was determined by HPLC at
a wavelength of 214 nm. Molecular mass was determined by MALDI-MS or ESI-TOF/MS (Applied Biosystems-Sciex
5800 MALDI/TOF/TOF-MS, Bruker BioTOF II ESI-TOF/MS, University of Minnesota Mass Spectometry Lab).

SNP Incorporations, Asn-Substituted Scaffold: Compound 1 (MDE 6-82-3c, SEQ ID NO:1), which contains the endogenous sequence of AgRP from residues 111-116 (Arg-Phe-Phe-Asn-Ala-Phe (SEQ ID NO: 41)), cyclized through a DPro-Pro motif, was previously reported to have antagonist activity at the mMC3R and mMC4R (pA₂=6.3 and 8.2 respectively), inverse agonist activity at the mMC5R, and partially stimulated the mMC1R (25% of NDP-MSH signal at 100 μM). (Ericson et al., J Med Chem, 58 (11): 4638-4647, 2015) In the present study, this compound was additionally found to possess antagonist activity at the hMC4R (pA₂=8.0) and mMC5R (pA₂=6.4), as well as inverse agonist activity at the hMC4R (Tables 2 and 3).

TABLE 2
Summary of the antagonist pA₂ and inverse agonist pharmacology of AgRP
macrocyclic analogues incorporating both single nucleotide polymorphism mutations
and Dap residues at the human melanocortin 4 receptor (hMC4R).^{a}

| | | | hMC4R | Observed Inverse |
|---|---|---|---|---|
| | Peptide | Sequence | pA₂ | Agonism? |
| | AgRP | | 8.7 ± 0.1 | Yes |
| ASN⁵ | 1* (MDE 6-82-3c) | c[Pro-Arg-Phe-Phe-Asn-Ala-Phe-DPro] | 8.0 ± 0.2 | Yes |
| | | SEQ ID NO: 1 | | |
| | AMK 2-82 | c[Pro-Arg-Phe-Phe-Asn-Val-Phe-DPro] | 7.8 ± 0.03 | Yes |
| | | SEQ ID NO: 3 | | |
| | ZMK 2-96 | c[Pro-His-Phe-Phe-Asn-Ala-Phe-DPro] | 5.8 ± 0.2 | Yes |
| | | SEQ ID NO: 4 | | |
| | ZMK 2-110 | c[Pro-Arg-Tyr-Phe-Asn-Ala-Phe-DPro] | 6.5 ± 0.2 | Yes |
| | | SEQ ID NO: 5 | | |
| | ZMK 3-18 | c[Pro-Cys-Phe-Phe-Asn-Ala-Phe-DPro] | 5.8 ± 0.2 | No |
| | | SEQ ID NO: 6 | | |
| DAP⁵ | 2* (MDE 6-82-1c) | c[Pro-Arg-Phe-Phe-Dap-Ala-Phe-DPro] | 8.6 ± 0.2 | Yes |
| | | SEQ ID NO: 2 | | |
| | ZMK 2-85 | c[Pro-Arg-Phe-Phe-Dap-Val-Phe-DPro] | 7.6 ± 0.06 | Yes |
| | | SEQ ID NO: 7 | | |
| | ZMK 2-99 | c[Pro-His-Phe-Phe-Dap-Ala-Phe-DPro] | 6.4 ± 0.09 | No |
| | | SEQ ID NO: 8 | | |
| | ZMK 2-112 | c[Pro-Arg-Tyr-Phe-Dap-Ala-Phe-DPro] | 7.5 ± 0.2 | Yes |
| | | SEQ ID NO: 9 | | |
| | ZMK 3-20 | c[Pro-Cys-Phe-Phe-Dap-Ala-Phe-DPro] | <5.0 | No |
| | | SEQ ID NO: 10 | | |

^{a}The indicated errors represent the standard error of the mean (SEM) determined from at least
three independent experiments. The antagonistic pA₂ values were determined using the Schild
analysis and the agonist NDP-MSH. >100,000 indicates that the compound was examined but lacked
agonist activity at up to 100 μM concentrations. <5.0 indicates that no antagonist potency was
observed in the highest concentration ranged assayed (10,000, 5,000, 1,000, and 500 nM).
Inverse agonist indicates that inverse agonist pharmacology was observed.
*The pharmacology for 1 and 2 has previously been reported.(Ericson et al., J Med Chem, 58 (11):
4638-4647, 2015)

TABLE 3
Summary of the cAMP based agonist EC₅₀ and antagonist pA₂ pharmacology values of AgRP
macrocyclic analogues incorporating both single mucleotide polymorphism
mutations and Dap residues at the mouse melanocortin receptors.^{a}

| | | | mMC1R | mMC3R | | mMC4R | | mMC5R | |
|---|---|---|---|---|---|---|---|---|---|
| | | | EC₅₀ | EC₅₀ | | EC₅₀ | | EC₅₀ | |
| | Peptide | Sequence | (nM) | (nM) | pA₂ | (nM) | pA₂ | (nM) | pA₂ |
| | NDP-MSH | | 0.010 ± | 0.07 ± | | 0.9 ± | | 0.13 ± | |
| | | | 0.003 | 0.03 | | 0.6 | | 0.03 | |
| ASN⁵ | 1* (MDE | c[Pro-Arg-Phe- | 25% @ | >100,000 | 6.3 ± 0.1 | >100,000 | 8.2 ± 0.1 | Inverse | 6.4 ± 0.2 |
| | 6-82-3c) | Phe-Asn-Ala- | 100 μM | | | | | Agonist | |
| | | Phe-DPro] | | | | | | −10% | |
| | | SEQ ID NO: 1 | | | | | | | |
| | ZMK 2-82 | c[Pro-Arg-Phe- | 23% @ | >100,000 | 5.9 ± 0.1 | 16% @ | 7.2 ± 0.1 | >100,000 | 6.1 ± 0.09 |
| | | Phe-Asn-Val- | 100 μM | | | 100 μM | | | |
| | | Phe-DPro] | | | | | | | |
| | | SEQ ID NO: 3 | | | | | | | |
| | ZMK 2-96 | c[Pro-His-Phe- | 23% @ | >100,000 | <5.0 | >100,000 | 5.3 ± 0.1 | >100,000 | N/A |
| | | Phe-Asn-Ala- | 100 μM | | | | | | |
| | | Phe-DPro] | | | | | | | |
| | | SEQ ID NO: 4 | | | | | | | |
| | ZMK | c[Pro-Arg-Tyr- | >100,000 | >100,000 | 5.8 ± 0.2 | >100,000 | 6.1 ± 0.03 | Inverse | N/A |
| | 2-110 | Phe-Asn-Ala- | | | | | | Agonist | |
| | | Phe-DPro] | | | | | | −35% @ | |
| | | SEQ ID NO: 5 | | | | | | 100 μM | |
| | ZMK 3-18 | c[Pro-Cys-Phe- | | | | | | | |
| | | Phe-Asn-Ala- | | | | | | | |
| | | Phe-DPro] | | | | | | | |
| | | SEQ ID NO: 6 | | | | | | | |
| DAP⁵ | 2* (MDE | c[Pro-Arg-Phe- | 30% @ | >100,000 | 6.5 ± 0.09 | >100,000 | 8.7 ± 0.1 | Inverse | 7.3 ± 0.03 |
| | 6-82-1c) | Phe-Dap-Ala- | 100 μM | | | | | Agonist | |
| | | Phe-DPro] | | | | | | −15% | |
| | | SEQ ID NO: 2 | | | | | | | |
| | ZMK 2-85 | c[Pro-Arg-Phe- | 79% @ | 27% @ | 6.1 ± 0.03 | 37% @ | 7.5 ± 0.1 | >100,000 | 6.3 ± 0.03 |
| | | Phe-Dap-Val- | 100 μM | 100 μM | | 100 μM | | | |
| | | Phe-DPro] | | | | | | | |
| | | SEQ ID NO: 7 | | | | | | | |
| | ZMK 2-99 | c[Pro-His-Phe- | 56% @ | >100,000 | 5.4 ± 0.1 | >100,000 | 6.0 ± 0.1 | >100,000 | N/A |
| | | Phe-Dap-Ala- | 100 μM | | | | | | |
| | | Phe-DPro] | | | | | | | |
| | | SEQ ID NO: 8 | | | | | | | |
| | ZMK | c[Pro-Arg-Tyr- | 48% @ | >100,000 | 6.5 ± 0.1 | >100,000 | 7.3 ± 0.1 | >100,000 | 6.5 ± 0.2 |
| | 2-112 | Phe-Dap-Ala- | 100 μM | | | | | | |
| | | Phe-DPro] | | | | | | | |
| | | SEQ ID NO: 9 | | | | | | | |
| | ZMK 3-20 | c[Pro-Cys-Phe- | 4000 ± | 29% @ | <5.0 | 42% @ | <5.0 | >100,000 | N/A |
| | | Phe-Dap-Ala- | 2000 | 100 μM | | 100 μM | | | |
| | | Phe-DPro] | | | | | | | |
| | | SEQ ID NO: 10 | | | | | | | |

^{a}The indicated errors represent the standard error of the mean (SEM) determined from at least three independent
experiments. The antagonistic pA₂ values were determined using the Schild analysis and the agonist NDP-MSH.
>100,000 indicates that the compound was examined but lacked agonist activity at up to 100 μM concentrations.
N/A indicates that the compound was not examined. A percentage denotes the percent maximal stimulatory
response observed at 100 μM concentrations but not enough stimulation was observed to determine an EC₅₀
value. <5.0 indicates that no antagonist potency was observed in the highest concentration ranged assayed
(10,000, 5,000, 1,000, and 500 nM). Inverse agonist indicates that inverse agonist pharmacology was observed
with the percent decrease from basal indicated. For inverse agonists, a decrease in cAMP signal was observed
without a sigmoidal dose-response curve, the percent change from basal at 100 μM concentration is indicated.
*The pharmacology for 1 and 2 has previously been reported, with the exception of the antagonistic
pA₂ values at the mMC5R which are reported herein for the first time.

This scaffold was designed to recreate the active loop of AgRP cyclized through a DPro-Pro motif that had previously been demonstrated to mimic β-hairpin conformations in other macrocyclic peptides. (Jiang et al., Helvetica Chimica Acta, 83 (12): 3097-3112, 2000) Therefore, this octapeptide scaffold was used to investigate four naturally occurring AgRP SNPs, without synthesizing the full-length peptide [46 residue AgRP(87-132)]. Substitution of the Ala115 with Val (rs773319622) resulted in ZMK 2-82 (SEQ ID NO:3). This peptide (ZMK 2-82) was able to partially stimulate the mMC1R (23% NDP-MSH signal at 100 μM), and was unable to stimulate the mMC3R, mMC4R, and mMC5R at the concentrations assayed. When assayed as an antagonist at the mMC3R, mMC4R, and mMC5R, ZMK 2-82 exhibited antagonist activity (pA₂=5.9, 7.2, and 6.1 respectively) and a pA₂ of 7.7 at the hMC4R. Replacing the Arg111 to a His (rs199927717) resulted in the peptide ZMK 2-96, which partially stimulated the MC1R (23% at 100 μM) and did not stimulate the mMC3R, mMC4R, or the mMC5R. ZMK 2-96 did not possess antagonist activity at the mMC3R (pA₂<5.0) and was a micromolar antagonist at the mMC4R (pA₂=5.3) and hMC4R (pA₂=5.8). Substituting the Phe112 with Tyr (rs200972106) resulted in ZMK 2-110, which was unable to stimulate the mMC1R, mMC3R, or mMC4R at the concentrations assayed, but possessed inverse agomst activity at the mMC5R (−35% at 100 μM), a pharmacology previously observed using this scaffold. (Ericson et al., J Med Chem, 60 (19):8103-8114, 2017; Fleming et al., ACS Chem Neurosci, 9 (5): 1141-1151, 2018) ZMK 2-110 was a micromolar to sub-micromolar antagonist at the mMC3R, the mMC4R. and the hMC4R (pA2=5.8, 6.1, and 6.5 respectively). The last peptide synthesized using this scaffold was ZMK 3-18, which contained an Arg111 to Cys (rs1012110755) substitution. This peptide (ZMK 3-18) partially stimulated the mMC1R (46% at 100 μM), was unable to stimulate the mMC3R or mMC4R, and possessed inverse agonist activity at the mMC5R (−32% at 100 μM). ZMK 3-18 did not have antagonist activity at the mMC3R or mMC5R (pA₂<5.0) but was a micromolar antagonist at the mMC4R (pA₂=5.9) and at the hMC4R (pA₂=5.8). Binding affinities reflected the functional activity data and ranged from micro to nanomolar affinities, depending on the SNP incorporated, at all receptors assayed. Binding data is summarized in Tables 4 and 5.

TABLE 4
Summary of binding IC₅₀ affinities using the ¹²⁵I-NDP-MSH orthosteric ligand, of AgRP
marcocyclic of analogues incorporating both single nucleotide polymorphism
mutations and Dap residues at the mouse melanocortin receptors (mMCRs).^{a}

| | | | mMC1R | | mMC3R | | mMC4R | | mMC5R | |
|---|---|---|---|---|---|---|---|---|---|---|
| | | | | Fold | | Fold | | Fold | | Fold |
| | | | IC₅₀ (nM) | Diff | IC₅₀ (nM) | Diff | IC₅₀ (nM) | Diff | IC₅₀ (nM) | Diff |
| | NDP-MSH | | 0.29 ± | | 8.2 ± 0.7 | | 1.30 ± 0.01 | | 140 ± 50 | |
| | | | 0.09 | | | | | | | |
| ASN⁵ | 1* (MDE | c[Pro-Arg- | 9000 ± | 1 | 17000 ± 5500 | 1 | 70 ± 20 | 1 | 12300 ± 800 | 1 |
| | 6-82-3c) | Phe-Phe-Asn- | 2000 | | | | | | | |
| | | Ala-Phe-DPro] | | | | | | | | |
| | | SEQ ID NO: 1 | | | | | | | | |
| | ZMK 2-82 | c[Pro-Arg- | 15000 ± | 2 | 53000 ± 11000 | 3 | 38 ± 2 | 2 | 29000 ± 3000 | 2 |
| | | Phe-Phe-Asn- | 8000 | | | | | | | |
| | | Val-Phe-DPro] | | | | | | | | |
| | | SEQ ID NO: 3 | | | | | | | | |
| | ZMK 2-96 | c[Pro-His- | >100,000 | | >100,000 | | 6700 ± 600 | 300 | >100,000 | |
| | | Phe-Phe-Asn- | | | | | | | | |
| | | Ala-Phe-DPro] | | | | | | | | |
| | | SEQ ID NO: 4 | | | | | | | | |
| | ZMK | c[Pro-Arg- | >100,000 | | >100,000 | | 2000 ± 100 | 100 | >100,000 | |
| | 2-110 | Tyr-Phe-Asn- | | | | | | | | |
| | | Ala-Phe-DPro] | | | | | | | | |
| | | SEQ ID NO: 5 | | | | | | | | |
| | ZMK 3-18 | c[Pro-Cys- | 24600 ± 100 | 3 | >100,000 | | 1900 ± 500 | 90 | >100,000 | |
| | | Phe-Phe-Asn- | | | | | | | | |
| | | Ala-Phe-DPro] | | | | | | | | |
| | | SEQ ID NO: 6 | | | | | | | | |
| DAP⁵ | 2* (MDE | c[Pro-Arg- | 5200 ± 300 | 1 | 3200 ± 600 | 1 | 1.9 ± 0.6 | 1 | 1700 ± 300 | 1 |
| | 6-82-1c) | Phe-Phe-Dap- | | | | | | | | |
| | | Ala-Phe-DPro] | | | | | | | | |
| | | SEQ ID NO: 2 | | | | | | | | |
| | ZMK 2-85 | c[Pro-Arg- | 8000 ± | 2 | 10000 ± 100 | 3 | 8.2 ± 0.8 | 4 | 2700 ± 100 | 2 |
| | | Phe-Phe-Dap- | 5000 | | | | | | | |
| | | Val-Phe-DPro] | | | | | | | | |
| | | SEQ ID NO: 7 | | | | | | | | |
| | ZMK 2-99 | c[Pro-His- | 25000 ± | 5 | 15000 ± 3000 | 5 | 1000 ± 300 | 500 | 12000 ± 6000 | 7 |
| | | Phe-Phe-Dap- | 6000 | | | | | | | |
| | | Ala-Phe-DPro] | | | | | | | | |
| | | SEQ ID NO: 8 | | | | | | | | |
| | ZMK | c[Pro-Arg- | 18000 ± | 4 | 28000 ± 12000 | 9 | 134 ± 7 | 70 | 36000 ± 2000 | 20 |
| | 2-112 | Tyr-Phe-Dap- | 1000 | | | | | | | |
| | | Ala-Phe-DPro] | | | | | | | | |
| | | SEQ ID NO: 9 | | | | | | | | |
| | ZMK 3-20 | c[Pro-Cys- | 33000 ± | 6 | 49000 ± 3000 | 15 | 10000 ± 2000 | 5200 | >100,000 | |
| | | Phe-Phe-Dap- | 2000 | | | | | | | |
| | | Ala-Phe-DPro] | | | | | | | | |
| | | SEQ ID NO: 10 | | | | | | | | |

^{a}The indicated errors represent the standard deviation (SD) determined from at least two independent experiments,
each consisting of duplicate replicates. >100,000 indicates that the compound was examined but lacked binding
affinity at up to 100 μM concentrations. In addition, NDP-MSH, c[Pro-Arg-Phe-Phe-Asn-Phe-DPro] (SEQ ID NO: 1),
and c[Pro-Arg-Phe-Phe-Dap-Ala-Phe-DPro] (SEQ ID NO: 2) are included as experimental controls and as reference
controls. The fold difference (fold diff) is determined between the peptide as compared to the
c[Pro-Arg-Phe-Phe-Asn-Ala-Phe-DPro] (SEQ ID NO: 1) or c[Pro-Arg-Phe-Phe-Dap-Ala-Phe-DPro] (SEQ ID NO: 2)
controls respectively

TABLE 5
Summary of binding affinity IC₅₀ values using the ¹²⁵I-NDP-MSH orthosteric
agonist ligand to competitively displace the AgRP bases macrocyclic
analogues incorporating both single nucleotide
polymorphism mutations and Dap residues at the hMC4R.^{a}

| | | | hMC4R | |
|---|---|---|---|---|
| | Peptide | Sequence | IC₅₀ (nM) | Fold Diff |
| | NDP-MSH | | 10 ± 2 | 1 |
| ASN⁵ | 1* (MDE 6-82-3c) | c[Pro-Arg-Phe-Phe-Asn-Ala-Phe-DPro] | 70 ± 20 | 1 |
| | | SEQ ID NO: 1 | | |
| | ZMK 2-82 | c[Pro-Arg-Phe-Phe-Asn-Val-Phe-DPro] | 250 ± 40 | 3 |
| | | SEQ ID NO: 3 | | |
| | ZMK 2-96 | c[Pro-His-Phe-Phe-Asn-Ala-Phe-DPro] | 22000 ± 2000 | 300 |
| | | SEQ ID NO: 4 | | |
| | ZMK 2-110 | c[Pro-Arg-Tyr-Phe-Asn-Ala-Phe-DPro] | 14300 ± 1200 | 2000 |
| | | SEQ ID NO: 5 | | |
| | ZMK 3-18 | c[Pro-Cys-Phe-Phe-Asn-Ala-Phe-DPro] | 19300 ± 1400 | 300 |
| | | SEQ ID NO: 6 | | |
| DAP⁵ | 2* (MDE 6-82-1c) | c[Pro-Arg-Phe-Phe-Dap-Ala-Phe-DPro] | 7.5 ± 0.4 | 1 |
| | | SEQ ID NO: 2 | | |
| | ZMK 2-85 | c[Pro-Arg-Phe-Phe-Dap-Val-Phe-DPro] | 32.2 ± 0.5 | 4 |
| | | SEQ ID NO: 7 | | |
| | ZMK 2-99 | c[Pro-His-Phe-Phe-Dap-Ala-Phe-DPro] | 2700 ± 600 | 400 |
| | | SEQ ID NO: 8 | | |
| | ZMK 2-112 | c[Pro-Arg-Tyr-Phe-Dap-Ala-Phe-DPro] | 1016 ± 9 | 100 |
| | | SEQ ID NO: 9 | | |
| | ZMK 3-20 | c[Pro-Cys-Phe-Phe-Dap-Ala-Phe-DPro] | 15700 ± 1400 | 2100 |
| | | SEQ ID NO: 10 | | |

^{a}The indicated errors represent the standard deviation determined from at least two independent
experiments, each consisting of duplicate replicates. >100,000 indicates that the compound was
examined but lacked binding affinity at up to 100 μM concentrations. NDP-MSH,
c[Pro-Arg-Phe-Phe-Asn-Ala-Phe-DPro] (SEQ ID NO: 1), andc[Pro-Arg-Phe-Phe-Dap-Ala-Phe-DPro]
(SEQ ID NO: 2) are included as experimental controls and reference controls. The fold difference
(fold diff) is determined between the peptide as compared to the c[Pro-Arg-Phe-Phe-Asn-Ala-Phe-DPro]
(SEQ ID NO: 1) or c[Pro-Arg-Phe-Phe-Dap-Ala-Phe-DPro] (SEQ ID NO: 2) controls respectively.

SNP Incorporations, Dap-Substituted Scaffold: Compound 2 (MDE 6-82-1c, SEQ ID NO:2) incorporates the basic amino acid diaminopropionic acid (Dap) at the Asn⁵ position in the sequence c[-Pro-Arg-Phe-Phe-Asn⁵-Ala-Phe-DPro] (SEQ ID NO: 1) and has been previously reported to be an equipotent antagonist to endogenous AgRP antagonist at the MC4R. (Li, et al., Int J Obes (Lond) 38: 724-729, 2014) Due to the increased potency of 2, as compared to 1, it was hypothesized that the potential losses in potency from the SNPs might be better detected in compound 2 as it and AgRP have similar staring potencies than the less potent compound 1. This macrocycle (2) was able to partially stimulate the mMC1R (30%), was unable to stimulate the mMC3R and mMC4R within the assayed concentrations, and possessed inverse agonist activity at the mMC5R and hMC4R. Compound 2 possessed antagonist activity at the mMC3R, mMC4R, mMC5R. and hMC4R with pA₂ values of 6.5, 8.7, 7.3, and 8.6 respectively. Replacement of Ala115 (AGRP numbering) with Val resulted in the compound ZMK 2-85. This peptide (ZMK 2-85) was able to partially stimulate the mMC1R (79% of maximal NDP-MSH signal at 100 μM), the mMC3R (27%), and the mMC4R (37%), and was unable to stimulate the mMC5R ZMK 2-85 also possessed antagonist activity at the mMC3R mMC4R, mMC5R and hMC4R (pA₂=6.1, 7.5, 6.3, and 7.6 respectively). Substitution of the Arg111 (AGRP numbering) with a His residue produced ZMK 2-99, which partially activated the mMC1R (56% at 100 μM) and was unable to stimulate the mMC3R, mMC4R, or the mMC5R. ZMK 2-99 was a micromolar antagonist at the mMC3R (pA₂=5.4) and mMC4R (pA₂=6.0) and a sub-micromolar antagonist at the hMC4R (pA₂=6.4). Converting the Phe at the 112 position (AGRP numbering) to Tyr, coupled with the Dap substitution resulted in ZMK 2-112. This peptide (ZMK 2-112) partially stimulated the mMC1R (48@100 μM), was unable to stimulate any other receptor assayed, and possessed sub-micromolar antagonist potency at the mMC3R, mMC4R, mMC5R, and hMC4R (pA₂=6.5, 7.3, 6.5, and 7.5 respectively). The last peptide contained an Arg111 (AGRP numbering) to Cys substitution (ZMK 3-20). ZMK 3-20 was the only peptide that acted as a full agonist at the mMC1R, stimulating the receptor to 98% of the NDP-MSH control, with an EC₅₀ of 4000±2000 nM. ZMK 3-20 partially stimulated the mMC3R (29%@ 100 μM) and mMC4R (42% @100 μM) but was unable to stimulate the mMC5R or the hMC4R up to 100 μM concentrations. This peptide did not have any apparent antagonist activity (pA₂<5.0) at the mMC3R, the mMC4R. or the hMC4R at the highest concentrations of antagonists assayed (10 μM). All Dap-containing peptides, in general, possessed more potent binding ICs affinities at the receptors assayed compared to their Asn-containing counterparts, which mirrored the cAMP functional data. Binding IC₅₀ affinities are reported in Tables 4 and 5.

### Discussion

This work, for the first time, explored human single nucleotide polymorphisms resulting in missense mutations in the purported binding loop of the endogenous AgRP antagonist that were identified from deposited information into the NIH Variation Viewer database. The protein AgRP antagonizes the MC3R and the MC4R in the central nervous system, in addition to functioning as an inverse agonist at the MC4R. These functions prevent the endogenous agonists from binding in the orthosteric receptor domain, which prompts an increase in food intake when administered in vivo. AgRP has been reported to have varied pharmacology at the MC5R, (Fong et al., Biochem Biophys Res Commun, 237 (3): 629-631, 1997: Ollmann et al., Science, 278 (5335): 135-138, 1997; Yang et al., Mol Endocrinol, 13 (1): 148-155, 1999) but any potential physiological roles of these data have not been reported. The database SNPs examined herein have not been previously explored to investigate their potential for altering cellular signaling and functional relevance at the MCRs. To examine the potential impact these SNPs have on cAMP signaling at the MCRs, they were incorporated into the octapeptide macrocyclic scaffold c[Pro-Arg-Phe-Phe-Xaa-Ala-Phe-DPro] (SEQ ID NO: 42), where Xaa is either Asn or Dap. This scaffold was previously reported to possess nanomolar potency (Asn) or equipotent potency (Dap) to AgRP (87-132) at the mMC4R. (Ericson et al., J Med Chem, 58 (11): 4638-4647, 2015) This scaffold was chosen for this study to incorporate the four polymorphisms due to its potency and synthetic amenability.

All compounds, except ZMK 2-110, were able to partially or fully stimulate the mMC1R consistent with prior work published utilizing this scaffold. (Ericson et al., J Med Chem. 58 (11): 46384647, 2015; Ericson et al., ACS Chem Neurosci, 9 (12): 3015-3023, 2018) All compounds tested resulted in at least a 10-fold decreased antagonist potency at the mMC4R and hMC4R, as compared to the respective control compounds 1 and 2 (Tables 2 and 3), suggesting that SNPs may impact AgRP function. The greatest loss of activity was seen in compounds with substitutions at the Arg position, decreasing potency by at least a 150-fold relative to the parent control compounds. The Arg position has been previously studied in the Arg-Phe-Phe pharmacophore of AgRP. (Tota et al., Biochemistry, 38 (3): 897-904, 1999) In a structure activity relationship study published on the homologous agouti (ASP) protein, which shares the Arg-Phe-Phe motif, Kiefer et al. reported the conclusion that charge contributed to activity at the Arg position, as binding increased as more positive substitutions were used Lys>His>Gln>Ala. (Kiefer et al., Biochemistry, 37 (4): 991-997, 1998) The His substitution may maintain a potential positive charge (depending upon local environmental pH), resulting in decreased potency. In the Arg to Cys polymorphisms, the Cys substitution may be disrupting the native disulfide bonds by introducing new disulfide bonds. Ten cysteine residues are found naturally in the carboxyl end of AgRP, which form five bonds, and create a “cysteine knot” structure. (Bolin et al., FEBS Lett, 451 (2): 125-131, 1999) Disruption of this structure could potentially alter the conformation of the protein and decrease its ability to bind properly, although the current scaffold does not permit the examination of this hypothesis, since it does not replicate the cysteines of the C-terminal domain. The substitution of Tyr at the Phe112 position exhibited a 110-fold loss in the Asn scaffold and a 25-fold loss in the Dap scaffold, as compared to their respective parent compounds. These findings are consistent with previously reported structure-activity studies using this scaffold, where either a complete loss in antagonist potency or a decrease in potency from the native sequence at the MC4R was described at the Phe112 position when unnatural aromatic amino acids were substituted, with the exception of a Nal(1′) substitution, which maintained the potency of the parent compound. (Ericson et al., J Med Chem, 58 (11): 4638-4647, 2015) The findings presented herein recapitulate the hypothesis in the field that the Arg-Phe-Phe sequence is critical for orthosteric binding and activity as previously reported. (Kiefer et al., Biochemistry. 37 (4): 991-997, 1998; Tota et al., Biochemistry, 38 (3): 897-904, 1999; Dinulescu et al., J Biol Chem, 275 (10): 6695-6698, 2000) Lastly, the Ala115 containing macrocyclic peptides resulted in 31- and 15-fold decreased antagonist potency. This position appears to be more amenable to substitutions, albeit resulting in a decreased response relative to control compounds. It has been postulated, based upon GPCR homology molecular modeling, that the ligand Ala side chain is juxtaposed a histidine receptor residue of the MC4R (H264, TM6), when AgRP is bound. (Chai et al., Biochemistry, 44 (9): 3418-3431, 2005) Previously reported ligand structure-activity studies have postulated that hydrophobic residues and polar residues (specifically Ser) substituted at the Ala115 position maintain at least micromolar antagonist potency (Ser exhibiting nanomolar potency) compared to the parent compound, but acidic residues Glu and Asp ablated antagonist activity at the MC4R. (Ericson et al., J Med Chem, 60 (19): 8103-8114, 2017)

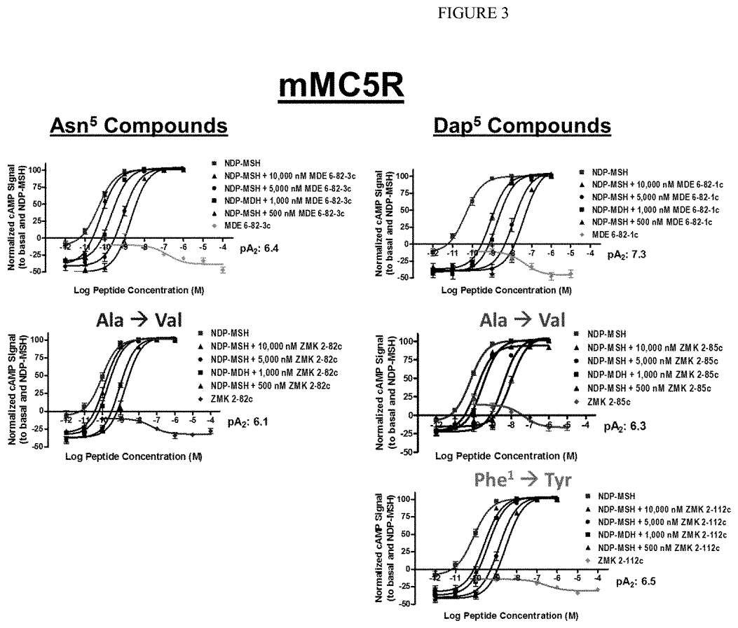

Many of the compounds were inactive when assayed as agonists at the mMC5R, apart from ZMK 2-110 and ZMK 3-18, which demonstrated inverse agonist activity, similar to the parent compounds as previously reported. (Ericson et al., J Med Chem, 58 (11): 46384647, 2015) Based upon the results from the binding studies (Table 4), demonstrating compounds that bound to the MC5R with micromolar binding IC₅₀ values, the 1* (MDE 6-82-3c), ZMK 2-82, ZMK 3-18, 2* (MDE 6-82-1c), ZMK 2-85, and ZMK 2-112 ligands were tested for MC5R antagonist pharmacology. The ZMK 2-82, ZMK2-85, and ZMK2-112 peptides possessed micromolar antagonist potencies at the mMC5R (pA₂=6.1, 6.3, and 6.5, respectively), suggesting that if AgRP has functional activity at the MC5R, these SNP may lead to altered MC5R signaling, FIG. 3.

This scaffold may therefore also serve as a lead in the further development of selective and potent MC5R antagonist ligands, which are lacking in the field to date. While the exact physiological role(s) of the ubiquitously expressed MC5R has not been elucidated, it has been linked to exocrine gland function in mice. (Chen et al., Cell, 91 (6): 789-798, 1997) Additionally, the MC5R has been investigated for its role in muscle glucose uptake (Enriori et al., Mol Metab, 5 (10): 807-822, 2016) and as a treatment for acne (reviewed by Zhang et al.). (Zhang et al., Eur J Pharmacol, 660 (1): 202-206, 2011) Non-selective MC5R antagonists have previously been reported, including HS024 that inhibits α-MSH-mediated cAMP stimulation at the MC1R, MC3R, MC4R, and MC5R when administered at 100 nM concentrations. (Kask et al., Endocrinology, 139 (12): 5006-5014, 1998) Many ligands have been reported to selectively bind to the MC5R, (Balse-Srinivasan et al., Journal of Medicinal Chemistry, 46 (17): 3728-3733, 2003: Cain et al., Bioorg Med Chem Lett, 16 (20): 5462-5467, 2006) although functional antagonist studies were not reported for these compounds. A peptidomimetic macrocycle (PG20N) was reported to be an MC5R antagonist (pA₂=8.3) that did not activate the MC1R and MC4R at concentrations up to 10 M and was reported to possess partial agonist activity at the MC3R (EC₅₀=50 nM; 21% the effect of MTII). (Grieco et al., J Med Chem, 51 (9): 2701-2707, 2008) PG20N was derived from the synthetic melanocortin agonist MT and antagonist SHU9119, while the library in the current study was based upon an AgRP template. Utilizing a different starting scaffold may allow the development of more potent and selective MC5R antagonist ligands that can be used to help determine the physiological roles of the MC5R in vivo.

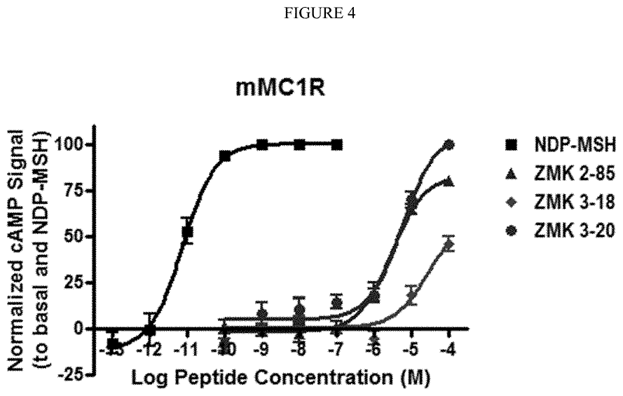

The two compounds that were the most active at the mMC1R (FIG. 4), ZMK 2-85 and ZMK 3-20, were also able to partially stimulate the mMC3R and mMC4R (27/37% and 29/42% of the maximal NDP-MSH signal respectively).

ZMK 2-85 possessed antagonist pharmacology at the mMC3R, mMC4R, and mMC5R (pA₂=6.1, 7.5, and 6.3 respectively). Compounds with this activity profile have been previously reported for the melanocortin system. (Hruby et al., J Med Chem, 38 (18): 3454-3461, 1995) Interestingly, ZMK 3-20 had no antagonist activity (pA₂<5.0) at the micromolar concentrations assayed at either the mMC3R or the mMC4R or the hMC4R but was a full micromolar agonist (4±2 μM) at the mMC1R and partially stimulated the mMC4R (42% of NDP-MSH at 100 μM). The Arg to Cys mutation, when incorporated with the basic residue Dap, produced a melanocortin ligand with unique pharmacology as compared to the rest of the library examined herein, which may be an important lead for studying the nuances of each receptor subtype and generating completely selective ligands.

From the findings in this study, it may be speculated that individuals with these polymorphisms express a form of AgRP that results in decreased MC4R potency, which may be ineffective at stimulating hunger, potentially resulting in a lean phenotype; however, the extent of any physiology consequences is not currently known and would need to be further experimentally verified. The scaffold used was an AgRP mimetic, therefore full-length AgRP incorporating the SNPs would need to be synthesized before concluding any physiologically based hypotheses relating the human SNPs and molecular function. There are many redundant mechanisms that nature has established to maintain energy homeostasis and satiety. For example, it has been reported that animals with genetic modifications preventing them from producing AgRP, NPY, or both, still maintain a normal weight, postulated to a result of compensatory pathways involved in the regulation of feeding and satiety homeostasis. (Qian et al., Mol Cell Biol, 22 (14): 5027-5035, 2002) Gropp et al. proposed this was likely due to the maintenance of the AgRP/NPY producing neurons in these animals, allowing for alternate compensatory pathways. When the neurons were ablated using diphtheria toxin, animals exhibited the expected phenotype within 24 hours, demonstrating that the AgRP/NPY neurons are mandatory for feeding behavior. (Gropp et al., Nat Neurosci. 8 (10): 1289-1291, 2005) However, these experiments represent situations where the signal (AgRP) is absent through neuronal ablation or genetic knockdown. In the case of the SNPs, the signal (AgRP) can transmit to the recipient (MC4R/MC3R), but at a potentially decreased potency. It is unknown what the human phenotype of this circumstance would be. It was apparent from the studies performed herein, that these polymorphisms altered the melanocortin receptor pharmacology from the endogenous sequence when tested in a live cell cAMP based functional assays and binding affinity assays and therefore should be further investigated in patients.

### Conclusions:

This study reports the first incorporation of deposited human SNPs of the binding loop of AgRP into a functional macrocyclic scaffold to determine the physiological consequences of the polymorphisms, as well as describes the first AgRP-derived mMC5R peptide antagonists. All compounds assayed demonstrated a decrease in antagonist potency at the MC4R compared to parent ligands of the wild-type sequence or the more potent Asn⁵Dap substitution scaffolds examined. These polymorphisms may represent a functional change when incorporated into the full-length AgRP, potentially altering AgRP signaling in individuals possessing these SNPs. At the MC1R, one compound did not possess agonist activity (ZMK 2-110), one ligand was a full agonist (ZMK 3-20), and the others partially stimulated it at 100 μM concentrations. Incorporation of the Dap residue at the 114 position (AGRP sequence numbering) did not universally increase MC4R potency (see ZMK 3-20) like previously reported. (Ericson et al., ACS Chem Neurosci, 9 (12): 3015-3023, 2018) Additionally, both SNPs that change Arg111 (to either His or Cys) saw the greatest decrease in potency, followed by the Phe112Tyr substitution, recapitulating the hypothesis in the field that the Arg-Phe-Phe(111-113 AGRP numbering) motif is critical for activity. Further phenotypic characterization of individuals possessing these SNPs will be important in determining the relevance of AgRP polymorphisms in the broader context of modulated energy homeostasis.

### Methods:

Peptide Synthesis: Peptides were synthesized using standard Fmoc chemistry. (Carpino et al., J Am Chem Soc, 92 (19): 5748, 1970; Carpino et al., Journal of Organic Chemistry, 37 (22): 3404, 1972) Coupling reagents 2-(1-H-benzotriazol-1-yl)-1,1,3,3-tetramethyluronium hexafluorophosphate (HBTU), benzotriazol-1-yl-oxy-tris(dimethylamino) phosphonium hexafluorophosphate (BOP), and 1-hydroxybenzotriazole (HOBt), the H-Pro-2-chlorotrityl resin, and Fmoc-protected amino acids were purchased from Peptides International (Louisville, Ky.). Dichloromethane (DCM), methanol (MeOH), acetonitrile (ACN), dimethylformamide (DMF) and anhydrous ethyl ether were purchased from Fisher (Fairlawn, N.J.). Trifluoroacetic acid (TFA), dimethyl sulfoxide (DMSO), piperidine, triisopropylsilane (TIS), and N,N-diisopropylethylamine (DIEA) were purchased from Sigma-Aldrich (St. Louis, Mo.). All reagents and chemicals were ACS grade or better and were used without further purification.

Peptides were synthesized on a 0.05 mmol scale using H-Pro-2-chlorotrityl resin (0.76 meq/g substitution) using a manual microwave synthesizer (CEM Discover SPS, Matthews, N.C.). Amino acids were added from carboxyl to amino terminal with two repeated steps consisting of Fmoc removal and amino acid coupling. Fmoc groups were removed with 20% piperidine in DMF for 2 min at room temperature and additionally for 4 min using microwave irradiation at 75° C. at 30 W. Incoming amino acids (3.1 eq) were installed with HBTU (3 eq) and DIEA (5 eq) with microwave irradiation at either 50° C. (His or Cys) or 75° C. for 5 min at 30 W. Arginine couplings required 5.1 eq of amino acid, 5 eq HBTU, and 7 eq of DIEA and an additional 5 minutes of irradiation time (10 min total). Deprotection and coupling steps were separated by at least four DMF washes and the ninhydrin (Kaiser et al., Anal Biochem, 34 (2) 595-598, 1970) or chloranil (for Proline) (Christensen, Acta Chemica Scandinavica Series B-Organic Chemistry and Biochemistry, 33 (10): 763-766, 1979) colorimetric assays were used to monitor reaction progress. After synthesis, peptides were cleaved from the resin, maintaining side-chain protecting groups using a 99:1 solution of DCM: TFA for a total of 6 min. The cleavage solution was concentrated under N₂ (g) and peptides were precipitated using ice-cold ethyl-ether. Side-chained protected peptides were cyclized head to tail forming an amide bond through the Arg and Pro residues, in DCM using BOP (3 eq) and HOBt (3 eq) at a peptide concentration of 0.33 mg/mL overnight. The DCM was subsequently removed under vacuum. Without further purification, side chain protecting groups were removed using a solution of a 95:2.5:2.5 solution of TFA:H₂O:TIS for 2 h. Peptide solution was concentrated under Nz (g) and cyclized peptide was precipitated using ice-cold ethyl-ether. Peptides were purified using RP-HPLC (Shimadzu) configured with a UV detector and a semi-preparative C18 bonded silica column (Vydac 218TP1010, 1×25 cm). Peptides were determined to have >95% purity before being assayed. Purity was determined by analytical RP-HPLC (Shimazdu) configured with a photodiode array detector and an analytical C18 silica column (Vydac 218TP104, 0.46×25 cm) in two different solvent systems. Molecular mass was determined by MALDI-MS or ESI-TOF/MS (Applied Biosystems-Sciex 5800 MALDI/TOF/TOF-MS, Bruker BioTOF II ESI-TOF/MS, University of Minnesota Mass Spectrometry Lab).

cAMP AlphaScreen® Bioassay: Compounds were dissolved in DMSO, apart from NDP-MSH, which was dissolved in H₂O, to a stock concentration of 10⁻² M. The compounds were characterized in HEK293 cells stably expressing the mMC1R, mMC3R, mMC4R, mMC5R, and hMC4R using a cAMP AlphaScreen® assay (Perkin Elmer), following the manufacturers' instructions and as previously described. (Ericson et al., J Med Chem, 58 (11): 4638-4647, 2015 Lensing et al., J Med Chem. 59 (7): 3112-3128, 2016) As the MC2R can only be stimulated by ACTH, it was excluded from this study. (Haynes et al., J Biol Chem, 225 (1): 115-124, 1957: Mountjoy et al., Science. 257 (5074): 1248-1251, 1992) Peptides were assayed first as agonists at the four mouse receptors. Compounds that did not elicit a full agonist response were then assayed as antagonists at the mouse MC3R, MC4R, and MC5R, and the human MC4R. Experiments were performed three independent times performed in duplicate.

To summarize, cells that had reached 75-85% confluency were removed with Versene (Gibco®) and plated at a concentration of 10,000 cells per well in a 384-well plate (Optiplate™), with 10 μL solution of prepared stimulation buffer (IX HBSS, 5 mM HEPES, 0.5 mM IBMX, 0.1% BSA, pH=7.4) and 0.5 μg of anti-cAMP acceptor beads, 5 μL of peptide containing solutions (concentrations ranging from 10⁻⁴ to 10⁻¹³, ligand potency dependent) was added to the wells to stimulate the cells and cells were incubated for 2 hours in the dark at room temperature. To determine the antagonist activity, cells were incubated with both the potent MCR agonist NDP-MSH (concentrations ranging from 10⁻⁶ to 10⁻¹²) and peptide (concentrations of 10,000 nM, 5,000 nM, 1,000 nM, and 500 nM), using a Schild paradigm. (Schild, Br J Pharmacol Chemother, 2 (3): 189-206, 1947) Following the incubation period, a 10 μL solution containing: 0.5 μg streptavidin coated donor beads, 0.62 μmol biotinylated-cAMP tracers, and lysis buffer (5 mM HEPES, 0.3% Tween-20, 0.1% BSA, pH=7.4) was added under green-light and incubated for an additional 2 h in the dark and at room temperature. Plates were read on an EnSpire® (Perkin Elmer) using the 384-Alpha plate pre-normalized assay protocol established by the manufacturer.

¹²⁵I-NDP-MSH Competitive Binding Assay: Peptide macrocycles were dissolved in DMSO and assayed using stably transfected HEK293 cells at the mMCRs and the hMC4R. The MC2R was excluded from this study. The resulting dose-response binding curves were used to determine the reported IC₅₀ values, using a protocol previously described. (Doering et al., J Med Chem, 60 (10): 4342-4357, 2017)

Briefly, cells were grown to >90% confluency in a 12-well polystyrene plate (Corning Life Sciences). Culture media was removed, and cells were incubated for 1 hour at 37° C., 5% CO₂ with 500 μL solution of 0.1% BSA in Dulbecco's Modified Eagle Medium (DMEM) containing the peptide macrocycles (concentrations ranging from 10⁻⁴ to 10⁻¹⁰ M) and a constant of 100,000 cpm/well ¹²⁵I-NDP-MSH. Post incubation, the media was removed, and the cells were washed and lysed with 500 μL of 0.1 M NaOH and 500 μL of 1% Triton X-100. Cells were incubated with the lysis solution for 10 minutes at room temperature before being transferred to 12 mm×75 mm polystyrene tubes. Radioactivity was quantified using a WIZARD2 automatic gamma counter (PerkinElmer). Specific binding was determined using nonradioactive NDP-MSH as a positive control, and the specific binding for compounds was normalized to 100% relative to NDP-MSH. Experiments were performed twice with duplicate wells in each experiment.

Data Analysis: The EC₅₀ and pA₂ values obtained from the AlphaScreen™ are representative of the means of duplicates assayed in at least three independent experiments. Compounds that did not possess agonist activity at the mMC3R, mMC4R, or mMC5R were screened as antagonists at these receptors and at the hMC4R. The EC₅₀ and pA₂ means and standard errors of the means (SEM) were determined using a non-linear least-squares analysis using PRISM software (v4.0, GraphPad Inc.) fit to the collected data. All peptides were assayed as TFA salts and not corrected for peptide weight. Inverse agonism was determined as a percent change from basal compared to the maximal signal of NDP-MSH at 100 μM. The IC₅₀ values obtained from the ¹²⁵I-NDP-MSH competitive binding assays are representative of the means of duplicates assayed in two independent experiments. The IC₅₀ means and standard deviations (SD) were determined using a non-linear least-squares analysis using PRISM software (v4.0, GraphPad Inc).

Homology models of human MC4R with peptide-based antagonists: In the absence of crystal structures of melanocortin receptors (MCRs), homology modeling remains a viable alternative for structural analysis of MCR-ligand complexes. In 2005 the first homology model was generated of the inactive conformation of hMC4R in complex with the C-terminal region of hAGRP (PDB ID:2iqv, 2iqr) (Chai et al., Biochemistry, 44 (9): 3418-3431, 2005) using distance geometry calculations by DIANA. (Guntert et al., Journal of Molecular Biology, 217 (3): 517-530, 1991) The model was based on crystal structures of bovine rhodopsin (PDB ID:Igzm) (Li et al., Journal of Molecular Biology, 343 (5): 1409-1438, 2004), which has only ˜15% sequence similarity to hMC4R, the NMR structure of hAGRP (PDB ID: 1hyk, residues 87-132) (McNulty et al., Biochemistry, 40 (51): 15520-15527, 2001), and experimentally-derived structural restraints. (Chai et al., Biochemistry, 44 (9): 3418-3431, 2005) Our subsequent modeling by distance geometry calculations in 2009 (Tan et al., Endocrinology 150 (1): 114-125, 2009) was based on the beta-2 adrenergic receptor structure (PDB ID: 2rh1) that has higher (˜30%) sequence similarity to hMC4R. Both 1 gzm- and 2rh1-based models were rather similar with rmsd 2.2 Å for 240 Cα-atoms, though they demonstrated some helix shift and deviations in intracellular (IL) and extracellular (EL) loop conformations. Structures of ILs generally followed the corresponding templates, EL2 represented just a short interhelical link in extended conformation, EL3 was restricted by two disulfide bridges connecting EL3 to TM6 (C271-C277) and to N-terminus (C40-C279) (Chai et al., Biochemistry, 44 (9): 3418-3431, 2005), while EL1 was either unfolded (in 2rh1-based model) or folded into a β-hairpin (in lgzm-based model).

At present, these models can be substantially updated due to the progress in structural determination of GPCRs and advances in computational methods for homology modeling. Currently, ˜50 crystal structures of GPCRs are available and can be used for homology modeling by automated servers, such as SWISS-MODEL (Biasini et al., Nucleic Acids Res, 42 (Web Server issue): W252-258, 2014), Phyre2 (Kelley et al., Nat Protoc, 10 (6): 845-858, 2015), RaptorX (Kallberg et al., Nat Protoc, 7 (8): 1511-1522, 2012), M4T (Fernandez-Fuentes et al., Nucleic Acids Res, 35 (Web Server issue): W363-368, 2007), and IntFOLD. (Roche et al., Nucleic Acids Res, 39 (Web Server issue): W171-176, 2011) Comparison of results of automated modeling of hMC4R by these servers demonstrated that crystal structures of human lysophospatidic acid receptor 1 (PDB ID: 4z35) (Chrencik et al., Cell, 161 (7): 1633-1643, 2015) and of the human sphingosine 1-phosphate receptor 1 (PDB ID: 3v2y) (Hanson et al., Science, 335 (6070): 851-855, 2012) likely represent the optimal templates, as they are evolutionary closer to MCRs with the highest (˜34%) sequence similarity to hMC4R (27-29% sequence identity) and, unlike other GPCR structures, lack the conserved Pro in the TM5, which is also absent of MCRs. This evolutionarily conserved Pro from TM5 produces a helical aneurism (an α-bulge with 5 residues per turn), which affects the position of the extracellular part of the TM5 and the conformation of the short EL2.

The latest modeling of the hMC4R (UniProtKB ID: P32245, residues 40-317) in the inactive conformation was based on the refinement of the 4z35-based model of hMC4R generated by SWISS-MODEL server. (Biasini et al., Nucleic Acids Res, 42 (Web Server issue): W252-258, 2014) The particular focus was made on the reformation of ELs, as these loops enclose the ligand binding pocket and likely participate in receptor-ligand interactions. (Yang et al., J Biol Chem, 274 (20): 14100-14106, 1999; Patel et al., Journal of Molecular Biology, 404 (1): 45-55, 2010) The procedure included remodeling of EL3 to satisfy disulfide bridge restraints (Chai et al., Biochemistry, 44 (9): 3418-3431, 2005) and of the EL1 to exclude helical bulges and interference with N-terminus attached to EL3 via disulfide, as well as the adjustment of side chains to produces their allowed conformations that lack mutual hindrances. The obtained model was refined by energy minimization (100 steps) with CHARMm force field implemented in QUANTA (Accerlys) using a dielectric constant (e) of 10 and the adopted-basis Newton-Raphson method. After minimization the rmsd between hMC4R model and its structural template (4z35) was 1.6 Å for 208 equivalent Cα-atoms. The 4z35-based model slightly deviated from previous models: rmsd with 1 gzm- and 2rhl-based models were 2.2 and 1.8 Å for 240 Cα-atoms, respectively.

### Abbreviations

ACTH, Adrenocorticotropin Hormone; AgRP, Agouti-Related Protein; ASP, Agouti Signaling Protein: GPCR. G-protein Coupled Receptor; MCR. Melanocortin Receptor; MC1R, Melanocortin 1 Receptor; MC3R, Melanocortin 3 Receptor; MC4R, Melanocortin 4 Receptor; MC5R, Melanocortin 5 Receptor; m, Mouse; h, Human; Fmoc, 9-fluorenylmethoxycarbonyl; cAMP, Cyclic 5′-Adenosine Monophosphate; MSH, Melanocyte Stimulating Hormone; POMC, Proopiomelanocortin: NDP-MSH, (4-Norleucine-7-D-Phenylalanine)-Ac-Ser-Tyr-Ser-Nle-Glu-His-DPhe-Arg-Trp-Gly-Lys-Pro-Val-NH₂ (SEQ ID NO: 44): Dap, Dianunopropionic acid; RP-HPLC, Reverse Phase—High Performance Liquid Chromatography; DMEM, Dulbecco's Modified Eagle Medium; SEM, Standard Error of the Mean: SD, Standard Deviation.

### Example 2 Substituting NPhe⁴ in AGRP-Based c[Pro¹-Arg²-Phe³-Phe⁴-Xxx⁵-Ala⁶-Phe⁷-DPro⁸] (SEQ ID NO: 45) Scaffolds Maintains MC4R Antagonist Potency

### Abstract

The melanocortin receptors are involved in numerous physiological functions and are regulated by agonists derived from the proopiomelanocortin gene transcript and two endogenous antagonists, agouti and agouti-related protein (AGRP). The key binding and functional determinant of AGRP, an MC3R and MC4R antagonist, is an Arg-Phe-Phe tripeptide sequence located on an exposed hexapeptide (Arg-Phe-Phe-Asn-Ala-Phe (SEQ ID NO: 41)) loop. It has previously been observed that cyclizing this sequence through a DPro-Pro motif (c[Pro¹-Arg²-Phe³-Phe⁴-Asn⁵-Ala⁶-Phe⁷-DPro⁸] (SEQ ID NO: 1)) resulted in a macrocyclic scaffold with MC4R antagonist activity, with increased MC4R potency when a diaminopropionic acid (Dap) residue is substituted at position 5. In this report, a series of II single-peptoid substitutions were performed in the AGRP-derived macrocycles. While most peptoid substitutions decreased MC4R antagonist potency, it was observed that NPhe⁴ or NDab⁵ (diaminobutyric acid) maintained MC4R antagonist potency. The NPhe⁴ substitutions also resulted in MC5R antagonist activity equipotent to the parent scaffolds. These data may be used in the design of future MC4R and MC5R antagonist leads and probes that possess increased metabolic stability due to the presence of peptoid residues.

### Introduction

The melanocortin system consists of 5 receptors (Chhajlani et al., FEBS Letters, 309 (3): 417-420, 1992; Mountjoy et al., Science, 257 (5074); 1248-1251, 1992; Chhajlani et al., Biochemical and Biophysical Research Communications, 195 (2): 866-873, 1993: Gantz et al., Journal of Biological Chemistry, 268 (11): 8246-8250, 1993: Gantz et al., Journal of Biological Chemistry, 268 (20): 15174-15179, 1993; Roselli-Rehfuss et al., Proceedings of the National Academy of Sciences of the United States of America, 90 (19): 8856-8860, 1993: Gantz et al., Biochemical and Biophysical Research Communications, 200 (3): 1214-1220, 1994: Griffon et al., Biochemical and Biophysical Research Communications, 200 (2): 1007-1014, 1994) known to date from the GPCR superfamily, agonists including ACTH as well as α-, β-, and γ-MSH derived from the proopiomelanocortin gene transcript, (Nakanishi et al., Nature, 278 (5703): 423-427, 1979) and antagonists agouti and agouti-related protein (AGRP). (Lu et al., Nature, 371(6500):799-802, 1994; Willard et al., Biochemistry, 34 (38): 12341-12346, 1995: Ollmann et al., Science, 278 (5335): 135-138, 1997) Due to the central role of the melanocortin-3 and -4 receptors (MC3R & MC4R) in the regulation of appetite, weight, and energy homeostasis, (Fan et al., Nature, 385 (6612): 165-168, 1997; Huszar et al., Cell, 88 (1): 131-141, 1997: Butler et al., Endocrinology, 141 (9): 3518-3521, 2000; Chen et al., Nature Genetics, 26 (1): 97-102, 2000; Irani et al., European Journal of Pharmacology, 660 (1): 80-87, 2011) and that administration of MC3R and MC4R antagonist molecules increase food intake in rodents, (Fan et al., Nature, 385 (6612): 165-168, 1997: Ebihara et al., Diabetes, 48 (10): 2028-2033, 1999: Irani et al., European Journal of Pharmacology 66W (1): 80-87, 2011) developing MC3R and MC4R antagonist ligands may lead to new therapeutic lead compounds for the treatment of negative energy disorders including cachexia, anorexia, and failure to thrive. Since AGRP is a naturally occurring nanomolar to sub-nanomolar potent MC3R and MC4R antagonist(Ollmann et al., Science, 278 (5335): 135-138, 1997; Wilczynski et al., Journal of Medicinal Chemistry, 47 (9): 2194-2207, 2004) and MC4R inverse agonist, (Haskell-Luevano et al., Regulatory Peptides, 99 (1): 1-7, 2001: Nijenhuis et al., Molecular Endocrinology, 15 (1): 164-171, 2001) probes derived from AGRP may be useful in developing compounds that increase appetite.

Although potent, the relatively large size (50 residues) of the in vivo active form of AGRP (Creemers et al., Endocrinology, 147 (4): 1621-1631, 2006) may limit its ability to be optimized as a therapeutic lead. An Arg-Phe-Phe tripeptide sequence within AGRP has been identified to be critical for receptor binding. (Tota et al., Biochemistry, 38 (3): 897-904, 1999) Structural NMR studies performed on AGRP or a 34-residue “mini-AGRP” suggested this tripeptide sequence was part of an exposed β-hairpin loop composed of the hexapeptide sequence Arg-Phe-Phe-Asn-Ala-Phe (SEQ ID NO: 41). (Bolin et al., FEBS Letters, 451 (2):125-131, 1999; McNulty et al., Biochemistry, 40 (51): 15520-15527, 2001: Jackson et al., Biochemistry, 41 (24): 7565-7572, 2002) While many AGRP truncation studies led to ligands with decreased antagonist potencies, a scaffold consisting of the active-loop hexapeptide cyclized head-to-tail through a DPro-Pro motif (c[Pro¹-Arg²-Phe³-Phe⁴-Asn⁵-Ala⁶-Phe⁷-DPro⁸](SEQ ID NO: 1)) resulted in a ligand that was 50-fold less potent than AGRP at the MC4R. (Ericson et al., Journal of Medicinal Chemistry, 58 (11): 46384647, 2015) Further structure-activity relationship studies demonstrated that replacement of the Asn with a diaminopropionic (Dap) residue resulted in a octapeptide macrocycle (c[Pro¹-Arg²-Phe³-Phe⁴-Dap⁵-Ala⁶-Phe⁷-DPro⁸] (SEQ ID NO: 2)) that was equipotent to AGRP at the MC4R. (Ericson et al., Journal of Medicinal Chemistry, 58 (11): 4638-4647, 2015; Ericson et al., Journal of Medicinal Chemistry, 60 (19): 8103-8114, 2017) Setmelanotide, a cyclic octapeptide melanocortin agonist ligand [Ac-Arg-c(Cys-DAla-His-DPhe-Arg-Trp-Cys) (SEQ ID NO: 46)] has been reported to decrease self-reported hunger scores and decrease body weight in select patient populations, (Kuhnen et al., New England Journal of Medicine, 375 (3): 240-246, 2016: Clement et al., Nature Medicine, 24 551-555, 2018) suggesting melanocortin peptide ligands of this size may be optimized for therapeutic effects in vivo.

One strategy to improve the stability of peptide ligands is to replace amino acids with peptoid residues, in which an amino acid side chain is appended to the nitrogen atom of a glycine residue instead of the α-carbon. Pioneered by Zuckermann, (Simon et al., Proceedings of the National Academy ofSciences of the United States of America, 89 (20):9367-9371, 1992; Zuckermann et al., Journal ofthe American Chemical Society, 114 (26): 10646-10647, 1992) such oligo-N-substituted glycine chains have been demonstrated to possess increase proteolytic stability compared to the corresponding all-L amino acid peptides. (Miller et al., Bioorganic & Medicinal Chemistry Letters, 4 (22): 2657-2662, 1994) Melanocortin scaffolds with peptoid substitutions have been reported to possess nanomolar agonist activity (Ac-Gly-Phe-NPhe-Arg-Trp-Gly-NH₂ (SEQ ID NO: 47) at the MC4R) (Ovadia et al., Bioorganic & Medicinal Chemistry, 18 (2): 580-589, 2010) and sub-nanomolar binding affinity (Ac-c[Cys-His-DPhe-Cys]-N^{α}-guanidinylbutyl-Trp-NH₂ (SEQ ID NO: 48) at the MC4R), (Ying et al., Journal of Medicinal Chemistry, 49 (23): 6888-6896, 2006) although a general trend for decreased agonist activity has been reported for peptoid scans of the melanocortin agonist tetrapeptide Ac-His-DPhe-Arg-Trp-NH₂ (SEQ ID NO: 49)(Holder et al., Bioorganic & Medicinal Chemistry Letters, 13 (24): 4505-4509, 2003) or heptapeptide Ac-Nle-Gly-Lys-DPhe-Arg-Trp-Gly-NH₂ (SEQ ID NO: 50) sequences. (Kruijtzer et al., Journal of Medicinal Chemistry, 48 (13): 4224-4230, 2005) Work with a tripeptoid scaffold based upon the Arg-Phe-Phe active sequence of AGRP yielded a compound with micromolar binding affinity that was able to dose-dependently decrease α-MSH cAMP production at the MC4R using micromolar concentrations, a 1000-fold decrease compared to AGRP. (Thompson et al., Bioorganic & Medicinal Chemistry Letters. 13 (8): 1409-1413, 2003)

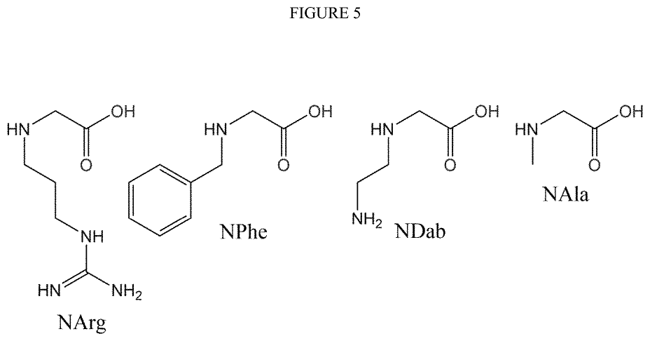

While peptoid substitutions in melanocortin ligands have trended towards decreased potency, with noted exceptions, it has been observed that incorporation of peptoid residues into a protegrin I antibiotic peptide permitted the peptide-peptoid ligand cyclized head-to-tail through a DPro-Pro motif to adopt a regular 0-hairpin conformation. (Shankaramma et al., Chemical Communications, (15): 1842-1843, 2003) Due to an observed β-hairpin conformation observed in the NMR structures of AGRP, (Bolin et al., FEBS Letters, 451 (2): 125-131, 1999; McNulty et al., Biochemistry, 40 (51): 15520-15527, 2001: Jackson et al., Biochemistry, 41 (24): 7565-7572, 2002) it was hypothesized that incorporation of peptoid residues into the AGRP octapeptide macrocyclic scaffold cyclized through a DPro-Pro motif might modulate potency. Using commercially available Fmoc-NPhe, Fmoc-NArg, and Fmoc-NAla amino acids (see FIG. 5 for structures of NArg, NPhe, NDab and DAla), single peptoid substitutions were investigated in the c[Pro¹-Arg²-Phe³-Phe⁴-Dap⁵-Ala⁶-Phe⁷-DPro⁸] (SEQ ID NO: 2) scaffold. To investigate the Dap position, an Fmoc-NDab (diaminobutyric acid, one methylene unit longer compared to Dap) residue was incorporated. Comparable peptoid insertions were also made in the c[Pro¹-Arg²-Phe³-Phe⁴-Asn⁵-Ala⁶-Phe⁷-DPro⁸] (SEQ ID NO: 1) scaffold to investigate how peptoid substitutions affected a similar set of compounds.

Peptide Synthesis and Characterization: Peptides were synthesized using standard Fmoc techniques, (Carpino et al., Journal of the American Chemical Society, 92 (19): 5748-5749, 1970 Carpino et al., Journal of Organic Chemistry. 37 (22): 3404-3409, 1972) as previously described. (Ericson et al., Journal of Medicinal Chemistry. 58 (11): 4638-4647, 2015; Ericson et al., Journal of Medicinal Chemistry, 60 (19): 8103-8114, 2017; Ericson et al., Journal of Medicinal Chemistry, 60 (2): 805-813, 2017; Ericson et al., ACS Chemical Neuroscience, DOI: 10.1021/acschemneuro.1028b00218, 2018) Due to incomplete coupling following the addition of the Fmoc-NPhe residue at select positions, the synthetic procedure was modified as noted in the experimental section. Peptides were purified to at least 95% purity in two diverse solvent systems and the correct average molecular weight was determined by ESI-MS or MALDI-MS (University of Minnesota Mass Spectrometry Lab). Purified cyclic peptides were dissolved in DMSO (10⁻² M) for pharmacological characterization at the mouse MC1R, MC3R, MC4R, and MC5R using the AlphaScreen cAMP assay. Peptides were assayed for agonist activity at all receptors. Compounds that did not possess agonist activity at the MC3R and MC4R were then assayed for antagonist activity using a Schild assay paradigm (Schild, British Journal of Pharmacology and Chemotherapy, 2 (3): 189-206, 1947) and the synthetic melanocortin agonist NDP-MSH. (Sawyer et al., Proceedings of the National Academy of Sciences of the United States of America, 77 (10): 5754-5758, 1980) For compounds that appeared to possess inverse agonist activity at the MC5R (decrease signal from basal activity), the percent change from basal activity was recorded. Compounds that decreased basal activity at the highest concentration assayed but did not result in a sigmoidal dose-response curve were reported as the percent decrease from basal activity at 100 μM. Since the AlphaScreen assay results in a loss of signal, activity curves were normalized to baseline and maximal NDP-MSH signal for illustrative purposes. Due to the inherent error with the assay in our lab, compounds that were within a three-fold potency range were considered equipotent.

TABLE 6
Analytical Data for Peptides Synthesized in this Study.

| | | Retention Time | | | mass spectral | |
|---|---|---|---|---|---|---|
| | | (min)^{a} | | M | analysis | Purity |
| Peptide | Sequence | System 1 | System 2 | (calc) | (M + 1) | % |
| MDE6-82-1c | c[Pro-Arg-Phe-Phe-Dap-Ala-PheDPro] | 17.5 | 28.5 | 948.5 | 949.4 | >98% |
| | SEQ ID NO: 2 | | | | | |
| MDE8-108-3c | c[Pro-NArg-Phe-Phe-Dap-Ala-PheDPro] | 16.1 | 24.9 | 948.5 | 949.4 | >96% |
| | SEQ ID NO: 23 | | | | | |
| MDE9-32c | c[Pro-DPro] | 16.4 | 25.6 | 948.5 | 949.6 | >99% |
| MDE9-93c | c[Pro-DPro] | 17.8 | 27.2 | 948.5 | 949.6 | >97% |
| MDE8-108-6c | c[Pro-DPro] | 16.5 | 25.9 | 948.5 | 949.5 | >99% |
| MDE8-108-7c | c[Pro-DPro] | 17.1 | 27.0 | 948.5 | 949.5 | >99% |
| MDE9-111c | c[Pro-DPro] | 17.7 | 26.4 | 962.5 | 963.7 | >97% |
| MDE7-26-1c | c[Pro-DPro] | 16.9 | 26.8 | 976.5 | 977.6 | >98% |
| MDE9-77c | c[Pro-DPro] | 17.5 | 27.1 | 976.5 | 977.7 | >99% |
| MDE9-88c | c[Pro-DPro] | 19.5 | 28.8 | 976.5 | 977.6 | >99% |
| MDE7-26-4c | c[Pro-DPro] | 18.6 | 28.3 | 976.5 | 977.7 | >97% |
| MDE7-26-5c | c[Pro-DPro] | 17.8 | 27.4 | 976.5 | 977.7 | >99% |

^{a}Peptide retention times (min) are reported for solvent system 1 (10% acetonitrile in 0.1% trifluoroacetic
acid/water and a gradient to 90% acetonitrile over 35 min) and solvent systme 2 (10% methanol in 0.1%
trifluoroacetic acid/water and a gradient to 90% methanol over 35 min). An analytical Vydac C18 column
(Vydac 218TP104) was used with a flow rate of 1.5 mL/min. The peptide purity was determined by HPLC at a
wavelength of 214 nm.

TABLE 7
Pharmacology of AGRP Macrocyclic Analogues at the Mouse Melanocortin Receptors.^{a}

| | | | mMC3R | | mMC4R | | mMC5R | |
|---|---|---|---|---|---|---|---|---|
| Peptide | Sequence | EC₅₀ (nM) | EC₅₀ (nM) | pA₂ | EC₅₀ (nM) | pA₂ | EC₅₀ (nM) | pA₂ |
| NDP- | | 0.010 ± | 0.09 ± 0.01 | | 0.9 ± 0.1 | | 0.11 ± 0.01 | |
| MSH | | 0.002 | | | | | | |
| MDE6- | c[Pro-Arg-Phe- | 36% @ | 20% @ | 6.5 ± 0.3 | >100,000 | 8.6 ± 0.1 | Inverse | 7.5 ± 0.1 |
| 82-1c | Phe-Dap-Ala- | 100 μM | 100 μM | | | | Agonist | |
| | Phe-DPro] | | | | | | −10% | |
| | SEQ ID NO: 2 | | | | | | | |
| MDE8- | c[Pro-NArg- | >100,000 | >100,000 | 5.9 ± 0.2 | >100,000 | 5.5 ± 0.2 | >100,000 | |
| 108-3c | Phe-Phe-Dap- | | | | | | | |
| | Ala-Phe-DPro] | | | | | | | |
| | SEQ ID NO: 23 | | | | | | | |
| MDE9- | c[Pro-Arg- | Partial | >100,000 | 6.9 ± 0.3 | >100,000 | 7.5 ± 0.1 | Partial | |
| 32c | NPhe-Phe-Dap- | Agonist | | | | | Agonist | |
| | Ala-Phe-DPro] | 80% NDP-MSH | | | | | 70% NDP-MSH | |
| | SEQ ID NO: 24 | 110 ± 50 | | | | | 550 ± 40 | |
| MDE9- | c[Pro-Arg-Phe- | Partial | >100,000 | 7.1 ± 0.1 | >100,000 | 9.0 ± 0.1 | Inverse | 7.4 ± 0.1 |
| 93c | NPhe-Dap-Ala- | Agonist | | | | | Agonist | |
| | Phe-DPro] | 60% NDP-MSH | | | | | −10% | |
| | SEQ ID NO: 12 | 2,700 ± 700 | | | | | | |
| MDE8- | c[Pro-Arg-Phe- | >100,000 | >100,000 | 5.6 ± 0.1 | >100,000 | 6.1 ± 0.1 | >100,000 | |
| 108-6c | Phe-Dap-NAla- | | | | | | | |
| | Phe-DPro] | | | | | | | |
| | SEQ ID NO: 25 | | | | | | | |
| MDE8- | c[Pro-Arg-Phe- | >100,000 | >100,000 | 5.5 ± 0.2 | >100,000 | 6.6 ± 0.1 | Inverse | |
| 108-7c | Phe-Dap-Ala- | | | | | | Agonist | |
| | NPhe-DPro] | | | | | | −10% | |
| | SEQ ID NO: 26 | | | | | | | |
| MDE9- | c[Pro-Arg-Phe- | 60% @ | >100,000 | 6.9 ± 0.1 | >100,000 | 8.4 ± 0.2 | Inverse | |
| 111c | Phe- [symbol] -Ala- | 100 μM | | | | | Agonist | |
| | Phe-DPro] | | | | | | −10% | |
| | SEQ ID NO: 27 | | | | | | | |
| MDE7- | c[Pro-NArg- | Partial | >100,000 | <5.5 | >100,000 | <5.5 | >100,000 | |
| 26-1c | Phe-Phe-Asn- | Agonist | | | | | | |
| | Ala-Phe-DPro] | 50% NDP-MSH | | | | | | |
| | SEQ ID NO: 28 | 3,100 ± 300 | | | | | | |
| MDE9- | c[Pro-Arg- | 240 ± 60 | >100,000 | 5.8 ± 0.1 | >100,000 | <5.5 | Inverse | |
| 77c | NPhe-Phe-Asn- | | | | | | Agonist | |
| | Ala-Phe-DPro] | | | | | | −10% @ | |
| | SEQ ID NO: 29 | | | | | | 100 μM | |
| MDE9- | c[Pro-Arg-Phe- | Partial | >100,000 | 6.0 ± 0.1 | >100,000 | 8.4 ± 0.1 | Inverse | 6.1 ± 0.1 |
| 88c | NPhe-Asn-Ala- | Agonist | | | | | Agonist | |
| | Phe-DPro] | 70% NDP-MSH | | | | | −10% | |
| | SEQ ID NO: 11 | 5,600 ± 200 | | | | | | |
| MDE7- | c[Pro-Arg-Phe- | 1,000 ± 200 | >100,000 | <5.5 | >100,000 | <5.5 | >100,000 | |
| 26-4c | Phe-Asn-NAla- | | | | | | | |
| | Phe-DPro] | | | | | | | |
| | SEQ ID NO: 30 | | | | | | | |
| MDE7- | c[Pro-Arg-Phe- | 85% @ | 60% @ | <5.5 | >100,000 | <5.5 | >100,000 | |
| 26-5c | Phe-Asn-Ala- | 100 μM | 100 μM | | | | | |
| | NPhe-DPro] | | | | | | | |
| | SEQ ID NO: 31 | | | | | | | |

^{a}The indicated errors represent the standard error of the mean determined from at least three independent experiments.
The use of >100,000 indicates that the compound was examined but lacked agonist activity at up to 100 μM
concentrations. A percentage denotes the percent maximal stimulatory response observed at 100 μM concentrations but
not enough stimulation was observed to determine an EC₅₀ value. Partial agonist indicates partial agonist activity
was observed, along with the percentage of activation relative to NDP-MSH and the EC₅₀ (compounds were considered
full agonist if >90% maximal NDP-MSH signal was observed). Inverse agonist indicates that inverse agonist pharmacology
was observed with the percent decrease from basal indicated. For inverse agonists, if a decrease in cAMP signal was
observed without a sigmoidal dose-response curve, the percentage change from basla at 100 μM concentrations is
indicated. The antagonistic pA₂ values were determined using the Schild analysis and the agonist NDP-MSH. The use
of <5.5 indicates that no antagonist potency was observed in the highest concentration ranged assayed (10,000, 5,000,
1,000, and 500 nM).

### Results and Discussion

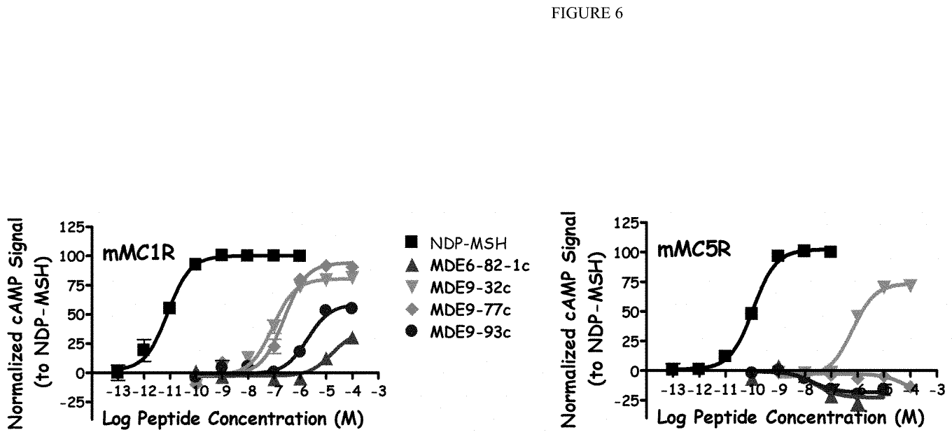

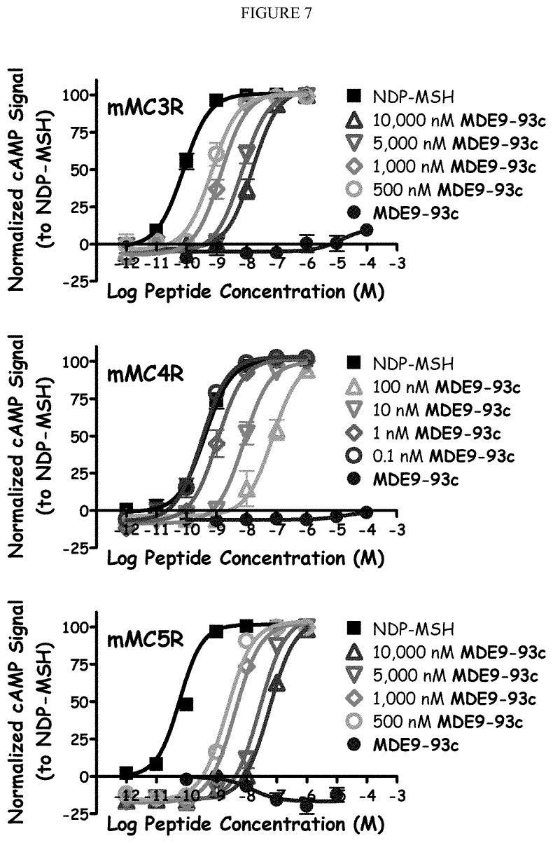

The lead macrocyclic peptide MDE6-82-1c (c[Pro¹-Arg²-Phe³-Phe⁴-Dap⁵-Ala⁶-Phe⁷-DPro⁸] (SEQ ID NO: 2)) possessed antagonist activity at the MC3R, MC4R. and MC5R (pA₂=6.5, 8.6, and 7.5, respectively), inverse agonist activity at the MC5R, and resulted in partial stimulation of the MC1R and MC3R at 100 μM concentrations. These values are similar to the antagonist potencies reported in Example 1 (pA₂ values of 6.5-7.2, 8.7-8.9, and 7.3 at the MC3R. MC4R, and MC5R, respectively) for this peptide, (Ericson et al., Journal of Medicinal Chemistry, 60 (19): 8103-8114, 2017; Fleming et al., Journal of Medicinal Chemistry, 61 (17): 7729-7740, 2018) and the inverse agonist pharmacology at the MC5R reported in Example 1. (Ericson et al., Journal of Medicinal Chemistry, 60 (19): 8103-8114, 2017; Fleming et al., ACS Chemical Neuroscience, 9 (5): 1141-1151, 2018) While substitution of NArg², NAla⁶, and NPhe⁷ (MDE8-108-3c, MDE8-108-6c, and MDE8-108-7c, respectively) resulted in no agonist activity at the MC1R, MC3R, and MC4R, substitution of NPhe³ (MDE9-32c) or NPhe⁴ (MDE9-93c) generated ligands that partially stimulated the MC1R (Table 7, FIG. 6 for MDE9-32c). Peptoid residues in this scaffold generated MC3R antagonist potencies (pA₂=5.5-7.1) in a similar range to the lead compound MDE6-82-1c (pA₂=6.5). While four of the five peptoid substitutions (NArg², NPhe³, NAla⁶, and NPhe⁷) decreased MC4R antagonist potency greater than 10-fold compared to the lead MDE6-82-1c (pA₂=8.6), substitution of NPhe⁴ (MDE9-93c) resulted in a pA₂ value of 9.0, the most potent observed in this study (FIG. 7). At the MC5R, two compounds did not possess agonist activity at the concentrations assayed (MDE8-108-3c and MDE8-108-6c), one compound partially stimulated the MC5R to 70% the maximal signal of NDP-MSH (MDE9-32c, FIG. 6), and two compounds possessed inverse agonist activity at the MC5R (MDE9-93c [FIGS. 8 and 9] and MDE8-108-7c). Due to MC4R antagonist potency and the MC5R inverse agonist activity. MDE9-93c was also evaluated for antagonist activity at the MC5R using a Schild assay paradigm, and was found to be an equipotent antagonist (pA₂=7.4, FIG. 7) compared to the MDE6-82-1c lead ligand at the MC5R.

In order to evaluate if a peptoid residue would be tolerated at the 5^{th} position, a commercially available NDab residue was inserted, yielding peptide-peptoid macrocyclic ligand MDE9-111c. This compound possessed similar pharmacology to the lead ligand (MDE6-82-1c), resulting in partial activation of the MC R at the highest concentrations assayed, sub-micromolar antagonist potency at the MC3R (pA₂=6.9), nanomolar antagonist potency at the MC4R (pA₂=8.4), and inverse agonist activity at the MC5R (Table 7).

A similar set of peptoid substitutions using NArg, NPhe, and NAla was also investigated in the c[Pro-Arg-Phe-Phe-Asn-Ala-Phe-DPro] (SEQ ID NO: 1) scaffold. At the MC1R, two compounds resulted in full agonist activity (MDE9-77c [FIG. 5] and MDE7-26-4c, with EC₅₀ values of 240 and 1,000 nM, respectively), two compounds were partial agonists (MDE7-26-1c and MDE9-88c), and one compound resulted in partial activation of the receptor at the highest concentrations assayed (MDE7-26-5c). Two compounds possessed micromolar antagonist potencies at the MC3R (MDE9-77c and MDE9-88c), while three (MDE7-26-1c, MDE-26-4c, and MDE7-26-5c) did not possess measurable MC3R antagonist activity in the concentrations assayed. One substitution, NPhe⁴ (MDE9-88c) possessed antagonist potency at the MC4R in the concentration range assayed (pA₂=8.4). The corresponding NPhe⁴ substitution in the Dap-scaffold (MDE9-93c) also resulted in the most potent MC4R antagonist compound in that scaffold. While three peptoid substitutions (MDE7-26-1c, MDE7-26-4c, and MDE7-26-5c) did not produce a response at the MC5R at up to 100 μM concentrations, one compound produced a decreased response from basal at the 100 μM concentrations (MDE9-77c, FIG. 6), and another (MDE9-88c) produced an inverse agonist response comparable to the lead ligand (MDE6-82-1c). As a comparison to the NPhe⁴, Dap MDE9-93c peptide, the antagonist potency of NPhe⁴, Asn⁵ MDE9-88c was measure at the MC5R (Table 7). This compound possessed sub-micromolar antagonist potency at the MC5R (pA₂=6.1), 20-fold decreased potency compared to MDE9-93c.

While past peptoid scans based upon agonist melanocortin sequences have resulted in decreased potencies, (Holder et al., Bioorganic & Medicinal Chemistry Letters, 13 (24): 4505-4509, 2003; Kruijtzer et al., Journal offedicinal Chemistry, 48 (13): 42244230, 2005) the present experiments demonstrate that positions 4 and 5 of the AGRP-derived macrocyclic scaffold c[Pro¹-Arg²-Phe³-Phe⁴-Xxx⁵-Ala⁶-Phe⁷-DPro⁸] (SEQ ID NO: 45) may be substituted with peptoid residues and maintain MC4R antagonist potency compared to the lead sequence. Substitution of NPhe at the Phe⁴ resulted in the most potent MC4R antagonist assayed with Dap at position 5 (MDE9-93c). The equivalent NPhe⁴ substitution when Asn was at position 5 (MDE9-8c) was the only Asn⁵ peptide-peptoid macrocycle to possess antagonist potency at the MC4R, and was equipotent to previous reports of the amino acid only form (reported pA₂ values at the MC4R range from 7.7-8.2). (Ericson et al., Journal of Medicinal Chemistry, 58 (11): 4638-4647, 2015; Ericson et al., Journal of Medicinal Chemisty, 60 (19): 8103-8114, 2017; Fleming et al., Journal of Medicinal Chemistry, 61 (17); 7729-7740, 2018) A previous report inverting the stereochemistry at this position in the macrocyclic scaffold, resulting in DPhe⁴, decreased antagonist potency greater than 10-fold. (Ericson et al., ACS Chemical Neuroscience, DOI: 10.1021/acschemneuro.1028b00218, 2018) It therefore appears that the loss of stereochemistry alone does not result in the increased potency observed for the NPhe substitutions. Antagonist potency at the MC4R is also maintained when a hPhe (the Phe sidechain is extended by one methylene unit) is inserted into position 4. (Ericson et al., Journal of Medicinal Chemistry. 58 (11): 46384647, 2015; Fleming et al., Journal of Medicinal Chemistry, 61 (17): 7729-7740, 2018) The extra length of the sidechain with a hPhe⁴ substitution may permit additional conformations of the side-chain that position the aromatic ring for more favorable interactions with the receptor. Shifting the side-chain from the α-carbon to the nitrogen may better position the NPhe⁴ aromatic ring in analogous conformations. Substitution of an NDab residue in position 5 (MDE9-111c) also maintained MC4R antagonist potency compared to the lead ligand MDE6-82-1c. This supports prior studies that a charge is important at this position, although the length of side chain, stereochemistry, and distribution of the charge (amine versus guanidinium group) is less important than the charge. (Ericson et al., Journal of Medicinal Chemistry, 58 (11): 46384647, 2015; Fleming et al., Journal of Medicinal Chemistry, 61 (17): 7729-7740, 2018)

As previously observed for the macrocyclic scaffolds, a Dap substitution at the 5^{th} position resulted in more potent MC4R antagonist potency compared to Asn. (Ericson et al., ACS Chemical Neuroscience, DOI: 10.1021/acschemneuro.1028b00218, 2018: Fleming et al., Journal of Medicinal Chemistry, 61 (17): 7729-7740, 2018) Peptoid replacement at the Arg², Phe³, Ala⁶ or Phe⁷ position all resulted in no observed antagonist activity at the MC4R when Asn was in the 5^{th} position, while micromolar to sub-micromolar antagonist potency was observed when Dap was in the 5^{th} position. For the NPhe⁴ peptoid substitution. Dap in the 5^{th} position remained more potent than Asn (pA₂=9.0 vs 8.4, respectively). These data support the previously observations that Dap increase MC4R antagonist potency and are useful to generate more potent MC4R antagonist ligands.

Unlike the observed activity at the MC4R, the two ligands that were observed to possess full MC1R agonist activity (MDE9-77c and MDE7-26-4c) had an Asn⁵. The equivalent Dap⁵ macrocycles with NPhe³ (MDE9-32c) and NAla⁶ (MDE8-108-6c) resulted in partial MC1R agonism (80% the maximal NDP-MSH signal) or no agonist activity at up to 100 μM concentrations, respectively. A D-amino acid scan of the Arg-Phe-Phe tripeptide sequence did not result in a similar agonist activity pattern at the MC R. (Ericson et al., ACS Chemical Neuroscience, DOI: 10.1021/acschemneuro.1028b00218, 2018) This observed MC1R agonism may suggest a peptoid substitution pattern that may be exploited in generated selective, potent MC1R agonist ligands.

Similar to prior reports, inverse agonist activity was observed for select ligands at the MC5R. (Ericson et al., Journal of Medicinal Chemistry, 60 (19): 8103-8114, 2017: Ericson et al., ACS Chemical Neuroscience, DOI: 10.1021/acschemneuro.1028b00218, 2018; Fleming et al., ACS Chemical Neuroscience, 9 (5): 1141-1151, 2018) Due to the increased MC4R antagonist potency, three compounds were examined for antagonist potency using a Schild experimental design at the MC5R, the parent MDE6-82-1c and the NPhe⁴ containing MDE9-93c and MDE9-88c. The parent MDE6-82-1c was reported in Example 1 to possess nanomolar MC5R antagonist potency (pA₂=7.3), similar to the value observed in the present study (pA₂=7.5). The NPhe⁴, Dap⁵ MDE9-93c resulted in an equipotent MC5R antagonist (pA₂=7.4) to MDE6-82-1c, indicating that peptoid substitution at this position was not detrimental to MC5R antagonism activity. A greater than 10-fold decrease in MC5R potency was observed comparing the parent MDE6-82-1c to the NPhe⁴, Asn⁵-containing MDE9-88c, similar to the drop observed in Example 1 between the Dap and Asn containing scaffolds without peptoid substitution (pA₂=7.3 and 6.4, respectively). These data suggest that the NPhe substitution does not negatively impact functional activity at the MC5R and may be useful in the future design of MC5R ligands that may be more enzymatically stable.

In this report, a series of single peptoid residues at Arg², Phe³, Phe⁴, Ala⁶, and Phe⁷ were substituted into macrocyclic peptide scaffolds possessing a Dap⁵ or Asn⁵ derived from the purported β-hairpin active loop of AGRP. Peptoid substitutions at the Arg², Phe³, Ala⁶, and Phe⁷ positions resulted in decreased MC4R antagonist potency, while equipotent activity was observed for NPhe⁴ substitution. Additionally, insertion of a basic peptoid residue (NDab) at position 5 also maintained MC4R antagonist activity, suggesting the 4^{th} and 5^{th} positions within the AGRP-derived macrocyclic scaffold are amendable to peptoid substitution. The NPhe⁴ substitutions also resulted in MC5R antagonist activity equipotent to the amino acid substituted macrocycles, suggesting the NPhe⁴ peptoid substitution was tolerated at the MCSR. These data may be useful in the future design of MC4R and MC5R antagonist ligands that may have increase enzymatic stability and serve as leads in the design of new probe molecules to interrogate the activity of the different melanocortin receptors in vivo.

### Experimental Methods:

The pre-loaded H-Pro-2Cl-Trityl resin, coupling reagents 2-(1H-benzotriazol-1-yl)-1,1,3,3-tetramethyluronium hexafluorophosphate (HBTU), 1-hydroxybenzotriazole (HOBt), and benzotriazol-1-yloxy-tris(dimethylamino)phosphonium hexafluorophosphate (BOP), and amino acids Fmoc-DPro, Fmoc-Phe, Fmoc-Ala, Fmoc-Dap(Boc), Fmoc-Asn(Trt), and Fmoc-Arg(Pbf) were purchased from Peptides International (Louisville, Ky.). The Fmoc-NPhe, Fmoc-NAla, Fmoc-NArg(Pbf), and Fmoc-NDab(Boc) residues were purchased from the PolyPeptide Group (San Diego, Calif.). Dichloromethane (DCM), methanol (MeOH), acetonitrile (MeCN), dimethylformamide (DMF), and anhydrous ethyl ether were purchased from Fisher (Fair Lawn, N.J.). Trifluoroacetic acid, dimethylsulfoxide (DMSO), piperidine, triisopropylsilane (TIS), N,N-diisopropylethylamine (DIEA), and 1-[bis(dimethylamino)methylene]-1H-1,2,3-triazolo[4,5-b]pyridinium 3-oxide hexafluorophosphate (HATU) were purchased from Millipore Sigma (St. Louis, Mo.). All reagents and chemicals were purchased as ACS grade or better and were used without further purification.

Peptide Synthesis: The linear, protected peptides were synthesized using standard Fmoc techniques (Carpino et al., Journal of the American Chemical Society, 92 (19): 5748-5749, 1970; Carpino et al., Journal of Organic Chemistry, 37 (22): 3404-3409, 1972) on a H-Pro-2-chlorotrityl resin (0.67 to 0.76 mequiv/g). Syntheses consisted of two repeated steps: (i) removal of the Fmoc group with 20% piperidine (1× at rt for 2 min, 1× at rt for 18 min) and (ii) coupling of the incoming Fmoc-protected residue (3.1 equiv), HBTU (3 equiv), and DIEA (5 equiv) at rt for 45 min. Higher amounts of the Fmoc-Arg(Pbf) amino acid (5.1 equiv), HBTU (5 equiv), and DIEA (7 equiv) were used when coupling Arg. It was observed that coupling Fmoc-Phe or Fmoc-Arg(Pbf) to an NPhe residue resulted in incomplete addition with the standard HBTU coupling. Therefore the protected, linear peptides corresponding to MDE9-32c and MDE9-77c were purified by RP-HPLC before the cyclization reaction using a Shimadzu system with a UV detector and a semipreparative RP-HPLC C18 bonded silica column (Vydac 218TP1010, 1.0 cm×25 cm). In the syntheses of MDE9-88c and MDE9-93c, the Fmoc-Phe residues following the addition of the NPhe peptoid residues were added with a double coupling synthetic strategy and HATU (3 equiv) as the coupling reagent. The addition of Fmoc-Arg(Pbf) in MDE9-88c and MDE9-93c was performed as previously described. Following removal of the terminal Fmoc group, linear peptides were cleaved from the resin with protecting groups intact with a 99:1 DCM:TFA cleavage solution for 6 min. Following concentrating the solution, the protected peptides were precipitated in ice-cold ethyl ether. Peptides were cyclized in DCM with BOP (3 equiv) and HOBT (3 equiv) overnight. Following removal of the DCM under vacuum, the cyclized peptides were side-chain deprotected using a 95:2.5:2.5 solution of TFA:H₂O:TIS for 2 hrs. Crude cyclized peptides were precipitated with ice-cold ethyl ether.

Cyclized peptides were purified by RP-HPLC using a Shimadzu system with a UV detector and semipreparative RP-HPLC C18 bonded silica column (Vydac 218TP1010, 1.0 cm×25 cm). The peptides were at least 95% pure as assessed by analytical RP-HPLC on a Shimadzu system attached to a photodiode array detector using a RP-HPLC C18 bonded silica column (Vydac 218TP104, 0.46 cm×25 cm) in two diverse solvent systems (MeOH and MeCN) and had the correct average molecular mass by MALDI-MS or ESI-MS (Applied Biosystems-Sciex 5800 MALDI/TOF/TOF-MS or Bruker BioTOF II ESI-TOF/MS, University of Minnesota Mass Spectrometry Lab).

cAMP AlphaScreen Bioassay: Macrocyclic peptide ligands were dissolved in DMSO at a stock concentration of 10⁻² M and were characterized pharmacologically using HEK293 cells stably expressing the mouse MC1R, MC3R, MC4R, and MC5R by the cAMP AlphaScreen assay (PerkinElmer) according to the manufacturer's instructions and as previously described. (Ericson et al., Boorganic & Medicinal Chemistry Letters, 25 (22): 5306-5308, 2015; Singh et al., ACS Medicinal Chemistry Letters, 6 (5): 568-572, 2015; Lensing et al., Journal of Medicinal Chemistry, 59 (7): 3112-3128, 2016) The synthetic control peptide NDP-MSH was dissolved in H₂O at a concentration of 10⁻⁴ M.

Briefly, cells 70-90% confluent were dislodged with Versene (Gibco®) at 37° C. and plated 10,000 cells/well in a 384-well plate (Optiplate™) with 10 μL freshly prepared stimulation buffer (IX HBSS, 5 mM HEPES, 0.5 mM IBMX, 0.1% BSA, pH=7.4) with 0.5 μg anti-cAMP acceptor beads per well. The cells were stimulated with the addition of 5 μL stimulation buffer containing peptide (a seven point dose-response curve was used starting at 10⁻⁴ to 10⁻⁷ M, determined by ligand potency) or forskolin (10⁻⁴ M) and incubated in the dark at room temperature for 2 h.

Following stimulation, streptavidin donor beads (0.5 μg) and biotinylated-cAMP (0.62 μmol) were added to the wells in a subdued light environment with 10 μL lysis buffer (5 mM HEPES, 0.3% Tween-20, 0.1% BSA, pH=7.4) and the plates were incubated in the dark at room temperature for an additional 2 h. Plates were read on a Enspire (PerkinElmer) Alpha-plate reader using a pre-normalized assay protocol (set by the manufacturer).

Data Analysis: The EC₅₀ and pA₂ values represent the mean of duplicate replicates performed in three independent experiments. The pA₂ and EC₅₀ estimates and associated standard errors (SEM) were determined by fitting the data to a nonlinear less-squares analysis using the PRISM program (version 4.0, GraphPad Inc.). When analyzing the inverse agonist activity at the MC5R, each replicate was normalized to the replicate signal at 10⁻¹⁰ M to observe change from basal activity. The percent inverse agonist activity was calculated from the normalized signal of three independent experiments. When a sigmoidal dose-response was observed, the percent inverse agonist activity reported was the change from basal to the plateau signal at high ligand concentrations. When inverse agonist activity was observed without a plateau at high concentrations, the percent inverse activity reported was the percent change from basal to signal at 100 μM concentrations. The peptides were assayed as TFA salts and not corrected for peptide content.

### Abbreviations

ACTH, Adrenocorticotropin hormone; Fmoc, 9-fluorenylmethoxycarbonyl; AGRP, Agouti-Related Protein; GPCR, G Protein-Coupled Receptor; cAMP, cyclic 5′-adenosine monophosphate; MC1R, Melanocortin-1 Receptor; MC2R, Melanocortin-2 Receptor: MC3R, Melanocortin-3 Receptor; MC4R, Melanocortin-4 Receptor: MC5R, Melanocortin-5 Receptor; MCR, Melanocortin Receptor: MSH, Melanocyte Stimulating Hormone: POMC, Proopiomelanocortin; α-MSH, Alpha-Melanocyte Stimulating Hormone; β-MSH, Beta-Melanocyte Stimulating Hormone; γ-MSH, Gamma-Melanocyte Stimulating Hormone; μM, Micromolar; NDP-MSH (4-Norleucine-7-D-Phenylalanine), Ac-Ser-Tyr-Ser-Nle-Glu-His-DPhe-Arg-Trp-Gly-Lys-Pro-Val-NH2 (SEQ ID NO: 44); Nle, norleucine; RP-HPLC, reverse-phase high-pressure liquid chromatography; SAR, structure-activity relationships; SEM, standard error of the mean.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A cyclic compound of formula I: 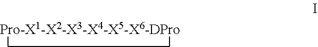
wherein:
Pro is a residue of L-proline;
X¹ is a residue of Arg, NArg, His or Cys;
X² is a residue of Phe, NPhe or Tyr;
X³ is a residue of Phe or NPhe;
X⁴ is a residue of Asn, Dap or NDab;
X⁵ is a residue of Ala, NAla or Val;
X⁵ is a residue of Phe or NPhe; and
DPro is a residue of D-proline;
or a salt thereof,
provided the compound of formula I is not c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2).

2. The compound of claim 1,
wherein:
Pro is a residue of L-proline;
X¹ is a residue of Arg;
X² is a residue of Phe or Tyr;
X³ is a residue of Phe or NPhe;
X⁴ is a residue of Asn or Dap;
X⁵ is a residue of Ala or Val;
X⁶ is a residue of Phe; and
DPro is a residue of D-proline;
or a salt thereof,
provided the compound of formula I is not c[Pro-Arg-Phe-Phe-Asn-Ala-Phe-DPro] (SEQ ID NO:1) or c[Pro-Arg-Phe-Phe-Dap-Ala-Phe-DPro] (SEQ ID NO:2).

3. The compound of claim 1, wherein X² is a residue of Tyr.

4. The compound of claim 1, wherein X² is a residue of Phe.

5. The compound of claim 1, wherein X³ is a residue of Phe.

6. The compound of claim 1, wherein X³ is a residue of NPhe.

7. The compound of claim 1, wherein X⁴ is a residue of Asn.

8. The compound of claim 1, wherein X⁴ is a residue of Dap.

9. The compound of claim 1, wherein X⁵ is a residue of Val.

10. The compound of claim 1, wherein X⁵ is a residue of Ala.

11. The compound of claim 1, which is selected from the group consisting of:
(SEQ ID NO: 3)
c[Pro-Arg-Phe-Phe-Asn-Val-Phe-DPro]
(SEQ ID NO: 4)
c[Pro-His-Phe-Phe-Asn-Ala-Phe-DPro]
(SEQ ID NO: 5)
c[Pro-Arg-Tyr-Phe-Asn-Ala-Phe-DPro]
(SEQ ID NO: 6)
c[Pro-Cys-Phe-Phe-Asn-Ala-Phe-DPro]
(SEQ ID NO: 7)
c[Pro-Arg-Phe-Phe-Dap-Val-Phe-DPro]
(SEQ ID NO: 8)
c[Pro-His-Phe-Phe-Dap-Ala-Phe-DPro]
(SEQ ID NO: 9)
c[Pro-Arg-Tyr-Phe-Dap-Ala-Phe-DPro]
(SEQ ID NO: 10)
c[Pro-Cys-Phe-Phe-Dap-Ala-Phe-DPro]
(SEQ ID NO: 11)
c[Pro-Arg-Phe-NPhe-Asn-Ala-Phe-DPro]
(SEQ ID NO: 12)
c[Pro-Arg-Phe-NPhe-Dap-Ala-Phe-DPro]
(SEQ ID NO: 23)
c[Pro-NArg-Phe-Phe-Dap-Ala-Phe-DPro]
(SEQ ID NO: 24)
c[Pro-Arg-NPhe-Phe-Dap-Ala-Phe-DPro]
(SEQ ID NO: 25)
c[Pro-Arg-Phe-Phe-Dap-NAla-Phe-DPro]
(SEQ ID NO: 26)
c[Pro-Arg-Phe-Phe-Dap-Ala-NPhe-DPro]
(SEQ ID NO: 27)
c[Pro-Arg-Phe-Phe- [symbol] -Ala-Phe-DPro]
(SEQ ID NO: 28)
c[Pro-NArg-Phe-Phe-Asn-Ala-Phe-DPro]
(SEQ ID NO: 29)
c[Pro-Arg-NPhe-Phe-Asn-Ala-Phe-DPro]
(SEQ ID NO: 30)
c[Pro-Arg-Phe-Phe-Asn-NAla-Phe-DPro]
(SEQ ID NO: 31)
c[Pro-Arg-Phe-Phe-Asn-Ala-NPhe-DPro]
and salts thereof.

12. The compound of claim 1, which is selected from the group consisting of:
(SEQ ID NO: 3)
c[Pro-Arg-Phe-Phe-Asn-Val-Phe-DPro]
(SEQ ID NO: 7)
c[Pro-Arg-Phe-Phe-Dap-Val-Phe-DPro]
(SEQ ID NO: 9)
c[Pro-Arg-Tyr-Phe-Dap-Ala-The-DPro]
(SEQ ID NO: 11)
c[Pro-Arg-Phe-NPhe-Asn-Ala-Phe-DPro]
(SEQ ID NO: 12)
c[Pro-Arg-Phe-NPhe-Dap-A1a-The-DPro]
and salts thereof.

13. The compound of claim 1, which is:
(SEQ ID NO: 3)
c[Pro-Arg-Phe-Phe-Asn-Val-Phe-DPro]
or a salt thereof.

14. The compound of claim 1, which is:
(SEQ ID NO: 7)
c[Pro-Arg-Phe-Phe-Dap-Val-Phe-DPro]
or a salt thereof.

15. The compound of claim 1, which is:
(SEQ ID NO: 9)
c[Pro-Arg-Tyr-Phe-Dap-Ala-Phe-DPro]
or a salt thereof.

16. The compound of claim 1, which is:
(SEQ ID NO: 11)
c[Pro-Arg-Phe-NPhe-Asn-Ala-Phe-DPro]
or a salt thereof.

17. The compound of claim 1, which is:
(SEQ ID NO: 12)
c[Pro-Arg-Phe-NPhe-Dap-Ala-Phe-DPro]
or a salt thereof.

18. A pharmaceutical composition comprising a compound of formula I as described in claim 1, or a pharmaceutically acceptable salt thereof, and a pharmaceutically acceptable carrier.

19. A method of modulating the activity of a melanocortin receptor in vitro or in vivo comprising contacting the receptor with an effective amount of a compound of formula I as described in claim 1, or a pharmaceutically acceptable salt thereof.

20. A method of modulating metabolic activity and/or modulating appetite in an animal in need thereof, comprising administering to the animal an effective amount of a compound of formula I as described in claim 1, or a pharmaceutically acceptable salt thereof.

21. A method for treating cachaxia or a disease associated with cachaxia in an animal in need thereof, comprising administering to the animal a compound of formula I as described in claim 1, or a pharmaceutically acceptable salt thereof.

22. A method of antagonizing MCR5 in an animal in need thereof, comprising administering to the animal an effective amount of a compound of formula I as described in claim 1, SEQ ID NO:1, or SEQ ID NO:2, or a pharmaceutically acceptable salt thereof.

23. A method for treating a disease or disorder associated with excessive MC5R activity in an animal in need thereof, comprising administering to the animal a compound of formula I as described in claim 1, SEQ ID NO:1, or SEQ ID NO:2, or a pharmaceutically acceptable salt thereof.


SEQUENCE LISTING (50 sequences)

SEQ ID NO: 1 - protein, 8 aa, Artificial Sequence
  Description of Artificial Sequence Synthetic peptide
  MOD_RES (8)..(8): DPro
PRFFNAFP

SEQ ID NO: 2 - protein, 8 aa, Artificial Sequence
  Description of Artificial Sequence Synthetic peptide
  MOD_RES (5)..(5): Dap
  MOD_RES (8)..(8): DPro
PRFFXAFP

SEQ ID NO: 3 - protein, 8 aa, Artificial Sequence
  Description of Artificial Sequence Synthetic peptide
  MOD_RES (8)..(8): DPro
PRFFNVFP

SEQ ID NO: 4 - protein, 8 aa, Artificial Sequence
  Description of Artificial Sequence Synthetic peptide
  MOD_RES (8)..(8): DPro
PHFFNAFP

SEQ ID NO: 5 - protein, 8 aa, Artificial Sequence
  Description of Artificial Sequence Synthetic peptide
  MOD_RES (8)..(8): DPro
PRYFNAFP

SEQ ID NO: 6 - protein, 8 aa, Artificial Sequence
  Description of Artificial Sequence Synthetic peptide
  MOD_RES (8)..(8): DPro
PCFFNAFP

SEQ ID NO: 7 - protein, 8 aa, Artificial Sequence
  Description of Artificial Sequence Synthetic peptide
  MOD_RES (5)..(5): Dap
  MOD_RES (8)..(8): DPro
PRFFXVFP

SEQ ID NO: 8 - protein, 8 aa, Artificial Sequence
  Description of Artificial Sequence Synthetic peptide
  MOD_RES (5)..(5): Dap
  MOD_RES (8)..(8): DPro
PHFFXAFP

SEQ ID NO: 9 - protein, 8 aa, Artificial Sequence
  Description of Artificial Sequence Synthetic peptide
  MOD_RES (5)..(5): Dap
  MOD_RES (8)..(8): DPro
PRYFXAFP

SEQ ID NO: 10 - protein, 8 aa, Artificial Sequence
  Description of Artificial Sequence Synthetic peptide
  MOD_RES (5)..(5): Dap
  MOD_RES (8)..(8): DPro
PCFFXAFP

SEQ ID NO: 11 - protein, 8 aa, Artificial Sequence
  Description of Artificial Sequence Synthetic peptide
  MOD_RES (4)..(4): NPhe
  MOD_RES (8)..(8): DPro
PRFFNAFP

SEQ ID NO: 12 - protein, 8 aa, Artificial Sequence
  Description of Artificial Sequence Synthetic peptide
  MOD_RES (4)..(4): NPhe
  MOD_RES (5)..(5): Dap
  MOD_RES (8)..(8): DPro
PRFFXAFP

SEQ ID NO: 13 - protein, 8 aa, Artificial Sequence
  Description of Artificial Sequence Synthetic peptide
  MOD_RES (8)..(8): DPro
PRFFNVFP

SEQ ID NO: 14 - protein, 8 aa, Artificial Sequence
  Description of Artificial Sequence Synthetic peptide
  MOD_RES (8)..(8): DPro
PHFFNAFP

SEQ ID NO: 15 - protein, 8 aa, Artificial Sequence
  Description of Artificial Sequence Synthetic peptide
  MOD_RES (8)..(8): DPro
PRYFNAFP

SEQ ID NO: 16 - protein, 8 aa, Artificial Sequence
  Description of Artificial Sequence Synthetic peptide
  MOD_RES (8)..(8): DPro
PCFFNAFP

SEQ ID NO: 17 - protein, 8 aa, Artificial Sequence
  Description of Artificial Sequence Synthetic peptide
  MOD_RES (5)..(5): Dap
  MOD_RES (8)..(8): DPro
PRFFXVFP

SEQ ID NO: 18 - protein, 8 aa, Artificial Sequence
  Description of Artificial Sequence Synthetic peptide
  MOD_RES (5)..(5): Dap
  MOD_RES (8)..(8): DPro
PHFFXAFP

SEQ ID NO: 19 - protein, 8 aa, Artificial Sequence
  Description of Artificial Sequence Synthetic peptide
  MOD_RES (5)..(5): Dap
  MOD_RES (8)..(8): DPro
PRYFXAFP

SEQ ID NO: 20 - protein, 8 aa, Artificial Sequence
  Description of Artificial Sequence Synthetic peptide
  MOD_RES (5)..(5): Dap
  MOD_RES (8)..(8): DPro
PCFFXAFP

SEQ ID NO: 21 - protein, 8 aa, Artificial Sequence
  Description of Artificial Sequence Synthetic peptide
  MOD_RES (4)..(4): NPhe
  MOD_RES (8)..(8): DPro
PRFFNAFP

SEQ ID NO: 22 - protein, 8 aa, Artificial Sequence
  Description of Artificial Sequence Synthetic peptide
  MOD_RES (4)..(4): NPhe
  MOD_RES (5)..(5): Dap
  MOD_RES (8)..(8): DPro
PRFFXAFP

SEQ ID NO: 23 - protein, 8 aa, Artificial Sequence
  Description of Artificial Sequence Synthetic peptide
  MOD_RES (2)..(2): NArg
  MOD_RES (5)..(5): Dap
  MOD_RES (8)..(8): DPro
PRFFXAFP

SEQ ID NO: 24 - protein, 8 aa, Artificial Sequence
  Description of Artificial Sequence Synthetic peptide
  MOD_RES (3)..(3): NPhe
  MOD_RES (5)..(5): Dap
  MOD_RES (8)..(8): DPro
PRFFXAFP

SEQ ID NO: 25 - protein, 8 aa, Artificial Sequence
  Description of Artificial Sequence Synthetic peptide
  MOD_RES (5)..(5): Dap
  MOD_RES (6)..(6): NAla
  MOD_RES (8)..(8): DPro
PRFFXAFP

SEQ ID NO: 26 - protein, 8 aa, Artificial Sequence
  Description of Artificial Sequence Synthetic peptide
  MOD_RES (5)..(5): Dap
  MOD_RES (7)..(7): NPhe
  MOD_RES (8)..(8): DPro
PRFFXAFP

SEQ ID NO: 27 - protein, 8 aa, Artificial Sequence
  Description of Artificial Sequence Synthetic peptide
  MOD_RES (5)..(5): NDab
  MOD_RES (8)..(8): DPro
PRFFXAFP

SEQ ID NO: 28 - protein, 8 aa, Artificial Sequence
  Description of Artificial Sequence Synthetic peptide
  MOD_RES (2)..(2): NArg
  MOD_RES (8)..(8): DPro
PRFFNAFP

SEQ ID NO: 29 - protein, 8 aa, Artificial Sequence
  Description of Artificial Sequence Synthetic peptide
  MOD_RES (3)..(3): NPhe
  MOD_RES (8)..(8): DPro
PRFFNAFP

SEQ ID NO: 30 - protein, 8 aa, Artificial Sequence
  Description of Artificial Sequence Synthetic peptide
  MOD_RES (6)..(6): NAla
  MOD_RES (8)..(8): DPro
PRFFNAFP

SEQ ID NO: 31 - protein, 8 aa, Artificial Sequence
  Description of Artificial Sequence Synthetic peptide
  MOD_RES (7)..(7): NPhe
  MOD_RES (8)..(8): DPro
PRFFNAFP

SEQ ID NO: 32 - protein, 8 aa, Artificial Sequence
  Description of Artificial Sequence Synthetic peptide
  MOD_RES (2)..(2): NArg
  MOD_RES (5)..(5): Dap
  MOD_RES (8)..(8): DPro
PRFFXAFP

SEQ ID NO: 33 - protein, 8 aa, Artificial Sequence
  Description of Artificial Sequence Synthetic peptide
  MOD_RES (3)..(3): NPhe
  MOD_RES (5)..(5): Dap
  MOD_RES (8)..(8): DPro
PRFFXAFP

SEQ ID NO: 34 - protein, 8 aa, Artificial Sequence
  Description of Artificial Sequence Synthetic peptide
  MOD_RES (5)..(5): Dap
  MOD_RES (6)..(6): NAla
  MOD_RES (8)..(8): DPro
PRFFXAFP

SEQ ID NO: 35 - protein, 8 aa, Artificial Sequence
  Description of Artificial Sequence Synthetic peptide
  MOD_RES (5)..(5): Dap
  MOD_RES (7)..(7): NPhe
  MOD_RES (8)..(8): DPro
PRFFXAFP

SEQ ID NO: 36 - protein, 8 aa, Artificial Sequence
  Description of Artificial Sequence Synthetic peptide
  MOD_RES (5)..(5): NDab
  MOD_RES (8)..(8): DPro
PRFFXAFP

SEQ ID NO: 37 - protein, 8 aa, Artificial Sequence
  Description of Artificial Sequence Synthetic peptide
  MOD_RES (2)..(2): NArg
  MOD_RES (8)..(8): DPro
PRFFNAFP

SEQ ID NO: 38 - protein, 8 aa, Artificial Sequence
  Description of Artificial Sequence Synthetic peptide
  MOD_RES (3)..(3): NPhe
  MOD_RES (8)..(8): DPro
PRFFNAFP

SEQ ID NO: 39 - protein, 8 aa, Artificial Sequence
  Description of Artificial Sequence Synthetic peptide
  MOD_RES (6)..(6): NAla
  MOD_RES (8)..(8): DPro
PRFFNAFP

SEQ ID NO: 40 - protein, 8 aa, Artificial Sequence
  Description of Artificial Sequence Synthetic peptide
  MOD_RES (7)..(7): NPhe
  MOD_RES (8)..(8): DPro
PRFFNAFP

SEQ ID NO: 41 - protein, 6 aa, Homo sapiens
RFFNAF

SEQ ID NO: 42 - protein, 8 aa, Artificial Sequence
  Description of Artificial Sequence Synthetic peptide
  MOD_RES (5)..(5): Asn or Dap
  MOD_RES (8)..(8): DPro
PRFFXAFP

SEQ ID NO: 43 - protein, 10 aa, Artificial Sequence
  Description of Artificial Sequence Synthetic peptide
  MOD_RES (3)..(3): Arg, Cys or His
  MOD_RES (4)..(4): Phe or Tyr
  MOD_RES (7)..(7): Ala or Val
YCXXFNXFCY

SEQ ID NO: 44 - protein, 13 aa, Artificial Sequence
  Description of Artificial Sequence Synthetic peptide
  MOD_RES (4)..(4): Nle
  MOD_RES (7)..(7): DPhe
SYSXEHFRWGKPV

SEQ ID NO: 45 - protein, 8 aa, Artificial Sequence
  Description of Artificial Sequence Synthetic peptide
  MOD_RES (5)..(5): Asn, Dap, or NDab
  MOD_RES (8)..(8): DPro
PRFFXAFP

SEQ ID NO: 46 - protein, 8 aa, Artificial Sequence
  Description of Artificial Sequence Synthetic peptide
  MOD_RES (3)..(3): DAla
  MOD_RES (5)..(5): DPhe
RCAHFRWC

SEQ ID NO: 47 - protein, 6 aa, Artificial Sequence
  Description of Artificial Sequence Synthetic peptide
  MOD_RES (3)..(3): NPhe
GFFRWG

SEQ ID NO: 48 - protein, 5 aa, Artificial Sequence
  Description of Artificial Sequence Synthetic peptide
  MOD_RES (3)..(3): DPhe
CHFCW

SEQ ID NO: 49 - protein, 4 aa, Artificial Sequence
  Description of Artificial Sequence Synthetic peptide
  MOD_RES (2)..(2): DPhe
HFRW

SEQ ID NO: 50 - protein, 7 aa, Artificial Sequence
  Description of Artificial Sequence Synthetic peptide
  MOD_RES (1)..(1): Nle
  MOD_RES (4)..(4): DPhe
XGKFRWG